SEQUENCE LISTING (6 sequences)

SEQ ID NO: 1 - DNA, 3594 bp, Homo sapiens
gctgtcagcgctgagcacagcgcccagggagagggacagacagccggctgcatgggacagcggaacccagagtgagaggggaggtggcaggacagacagacagcaggggcggacgcagagacagacagcggggacagggaggccgacacggacatcgacagcccatagattcctaacccagggagccccggcccctctcgccgcttcccaccccagacggagcggggacaggctgccgagcatcctcccacccgccctccccgtcctgcctcctcggcccctgccagcttcccccgcttgagcacgcagggcgtccgaggacgcgctgggcctccgcacccgccctcatggaggccgtggagaccggggaacggcccaccttcggagcctgggactacggggtctttgccctcatgctcctggtgtccactggcatcgggctgtgggtcgggctggctcggggcgggcagcgcagcgctgaggacttcttcaccgggggccggcgcctggcggccctgcccgtgggcctgtcgctgtctgccagcttcatgtcggccgtgcaggtgctgggcgtgccgtcggaggcctatcgctatggcctcaagttcctctggatgtgcctgggccagcttctgaactcggtcctcaccgccctgctcttcatgcccgtcttctaccgcctgggcctcaccagcacctacgagtacctggagatgcgcttcagccgcgcagtgcggctctgcgggactttgcagtacattgtagccacgatgctgtacaccggcatcgtaatctacgcaccggccctcatcctgaaccaagtgaccgggctggacatctgggcgtcgctcctgtccaccggaattatctgcaccttctacacggctgtgggcggcatgaaggctgtggtctggactgatgtgttccaggtcgtggtgatgctaagtggcttctgggttgtcctggcacgcggtgtcatgcttgtgggcgggccccgccaggtgctcacgctggcccagaaccactcccggatcaacctcatggactttaaccctgacccgaggagccgctatacattctggacttttgtggtgggtggcacgttggtgtggctctccatgtatggcgtgaaccaggcgcaggtgcagcgctacgtggcttgccgcacagagaagcaggccaagctggccctgctcatcaaccaggtcggcctgttcctgatcgtgtccagcgctgcctgctgtggcatcgtcatgtttgtgttctacactgactgcgaccctctcctcctggggcgcatctctgccccagaccagtacatgcctctgctggtgctggacatcttcgaagatctgcctggagtccccgggcttttcctggcctgtgcttacagtggcaccctcagcacagcatccaccagcatcaatgctatggctgcagtcactgtagaagacctcatcaaacctcggctgcggagcctggcacccaggaaactcgtgattatctccaaggggctctcactcatctacggatcggcctgtctcaccgtggcagccctgtcctcactgctcggaggaggtgtccttcagggctccttcaccgtcatgggagtcatcagcggccccctgctgggagccttcatcttgggaatgttcctgccggcctgcaacacaccgggcgtcctcgcgggactaggcgcgggcttggcgctgtcgctgtgggtggccttgggcgccacgctgtacccacccagcgagcagaccatgagggtcctgccatcgtcggctgcccgctgcgtggctctctcagtcaacgcctctggcctcctggacccggctctcctccctgctaacgactccagcagggcccccagctcaggaatggacgccagccgacccgccttagctgacagcttctatgccatctcctatctctattacggtgccctgggcacgctgaccactgtgctgtgcggagccctcatcagctgcctgacaggccccaccaagcgcagcaccctggccccgggattgttgtggtgggacctcgcacggcagacagcatcagtggcccccaaggaagaagtggccatcctggatgacaacttggtcaagggtcctgaagaactccccactggaaacaagaagccccctggcttcctgcccaccaatgaggatcgtctgtttttcttggggcagaaggagctggagggggctggctcttggaccccctgtgttggacatgatggtggtcgagaccagcaggagacaaacctctgaggacagggccagccgcgggactgacaccctgggatggaacctcaggatgggccaaacccagacaacgggcccatggccttgggctctgattggctggattgccttgtatgcaaatgagttcaggactacaataccctaccctatggggaggccctgcctccgggaggtcattttttaaatccagccccttgcttcaaccgtccccagtattagacgctgcagccctgacggctccccccaaataaggctgggtttttctctctctcttttttttttttttttttttttgagacagggtcatgctctgtcacccaggctggagtgcagtggtgtgatctcggctcactgcaacctctgcctcccaggctcaagtgattctcctgcctcagcctcttgagtagctgggattgtaggtgcccaccaccatgcccagccaactttttgtatttttagtagagacagggtttcaccatgttggtcaggctggtctcgaactcctgacctcaagtgatccacctgcctcggcctcccaaagtgctgggttacaggcgtaagctaccatgcccagcctaccgtttttctcaatctataatagaaagccaccacgcccaggtaatttttgtaattttgtatttttgtagagacggggttttgccatgttggccaggctggtctcaaactcctgatctcaggtgatcctcctgccttggcctcccaaagggctgggattacaggggtgagccaccgcgccccacctctttcttattttcttcctgggattggggaggggatgattcagaccccacatggcctccaaccttggcccacacacctgccatggctcccatcatcctgagcatgctagcgtcccctcctcacctgacaatggaggctctcgaattgggttgtgtccccccaaaatttatgtctacccagaacctcagaacatgagcttacttggaaatagggtctttgcaggtgtaactggttaaattaaaagaggtattactggaggaggatggatgaatccagtgactggttcctcatatgaagtagagaagagatgcagagaaacacatggggaagatgccacgtgaagacagaggcagtggttggatcaatgcatctacgagtcggagaacccaaggattgccagcaacaaccagaaatcaggaggggggcatgagatgcattatttcttagagcctttagagggaacatggccctactgacaccttgatgtcagacttctggctgctagaactgtcagagaataaatttctgttgtttgatgccaaaaaaaaaaaaaaaaaaa

SEQ ID NO: 2 - protein, 643 aa, Homo sapiens
MEAVETGERPTFGAWDYGVFALMLLVSTGIGLWVGLARGGQRSAEDFFTGGRRLAALPVGLSLSASFMSAVQVLGVPSEAYRYGLKFLWMCLGQLLNSVLTALLFMPVFYRLGLTSTYEYLEMRFSRAVRLCGTLQYIVATMLYTGIVIYAPALILNQVTGLDIWASLLSTGIICTFYTAVGGMKAVVWTDVFQVVVMLSGFWVVLARGVMLVGGPRQVLTLAQNHSRINLMDFNPDPRSRYTFWTFVVGGTLVWLSMYGVNQAQVQRYVACRTEKQAKLALLINQVGLFLIVSSAACCGIVMFVFYTDCDPLLLGRISAPDQYMPLLVLDIFEDLPGVPGLFLACAYSGTLSTASTSINAMAAVTVEDLIKPRLRSLAPRKLVIISKGLSLIYGSACLTVAALSSLLGGGVLQGSFTVMGVISGPLLGAFILGMFLPACNTPGVLAGLGAGLALSLWVALGATLYPPSEQTMRVLPSSAARCVALSVNASGLLDPALLPANDSSRAPSSGMDASRPALADSFYAISYLYYGALGTLTTVLCGALISCLTGPTKRSTLAPGLLWWDLARQTASVAPKEEVAILDDNLVKGPEELPTGNKKPPGFLPTNEDRLFFLGQKELEGAGSWTPCVGHDGGRDQQETNL

SEQ ID NO: 3 - DNA, 2943 bp, Mus musculus
ggtcgacggcccagcaggaactcgcgctgcgacgacgctcgctctgagagtccccgacgtcctccgcatcctctccgttccgagttacctgtctccatggagggcgcggaggcaggggcccgggccaccttcggcccctgggactacggcgtgttcgcgaccatgctgctggtgtccacgggcatcggtctgtgggtcggcctggcccgcggcggccagcgcagcgccgacgacttcttcaccgggggccggcagctggcggcagtgcctgtggggctgtcgctggccgctagcttcatgtcggctgtgcaggtgctcggggtccccgcggaggcagcgcgctacggtctcaagtttctgtggatgtgcgtgggccagttgctcaattcgctgctcacagccttgctcttcttgccgatcttctaccgcctgggccttaccagcacctaccagtacctagaactgcgcttcagccgagcggtccggctctgcgggacgctgcagtacttggtggccacgatgctgtatactggcatcgtgatctacgcacctgcgctcatcctgaaccaagtgaccgggttggacatctgggcatcgctcctgtccacaggaatcatctgcaccttgtacacgaccgtgggtggtatgaaggccgtggtctggacagatgtgttccaggttgtggtaatgctcgtcggcttctgggtgatcctggctcgaggtgtcatgctcatggggggcccctggaacgtgctcagtctcgctcaaaaccattcccggatcaacctgatggactttgaccctgaccctcggagccgctacaccttctggacatttgtagtaggtggctccctggtgtggctctccatgtatggtgtgaaccaagcccaggtgcagcgttatgtggcctgtcacacggagagaaaggccaagcttgccctgcttgtcaaccagcttggcctcttcctgattgtggctagtgcagcttgctgtggaattgtcatgtttgtctactacaaggactgcgaccccctcctcacaggccgcatcgcagcccccgatcagtacatgccattgctcgtgttggacatttttgaggatctgcccggagtccccgggctcttcctggcctgtgcctacagtggcaccctcagcaccgcgtccaccagtatcaacgctatggcagctgtaactgtggaagacctcatcaagcccaggatgcctagcctggcaccccggaagctggttttcatctctaaagggctctcattcatctatggctcaacctgcctcactgtggctgctctgtcctcgctgctgggaggtggggtcctccagggctctttcaccgtgatgggtgtcatcagtgggcctctccttggcgctttcaccctcgggatgctgctcccagcctgcaacacgccaggcgtcctctccgggctgacagcaggcttagctgtatccctgtgggtggccgtgggggccacgctgtacccgcctggagagcagaccatgggggtgctgcccacctcggctgcgggatgcaccaatgcctctgtcctcccgagcccacccggagctgccaacacttccagagggatccccagttctggaatggactcgggccgcccagcctttgccgacaccttttatgctgtctcctatctctactacggggctctgggtactttgaccactatgctttgtggtgctctcatcagctacctaactggccccaccaagcgcagctccctgggccccggattactgtggtgggacctcgctcggcagacagcatctgtggccccaaaggaggacaccaccaccctggaggacagcctggttaagggaccggaagacatccctgctgcgaccaagaagccccctggcttcaggccagaagccgagacccaccccctgtatctggggcacgatgctgagaccaacctctgagggcgaggcccgagaaagccaatcacaggcctcgggccagcagcctcctctctgaatggttggaccatcacctgtacagaagcttggctgatagaggccctgcccgccctgaagtccctgtgtcccacctgtgccccccaaaagagggttggttctctatccaccaaggaaaacatctggaaccgcagtgaccttgtagattgcagtaggcaactgagaacactcagcttctccagaccgtgaggttttcccatttaacaagcagagaagctgagggcggtcaccccaacgctgggaaggtagaggcaagagattcaggaggtcaagatcatccacagctacaggctgagaccctgcccacccccaaatatataaaagtaatctgggtgagccgcgtggcttcgcgacagagggcttgcccagcacacagaagacccaaggcagagggagaaatgggatgtccacagaacacagagccccaaggattgtgaagcttccgcgaagtgccaagggacagattctcagagccctcacaagacacggatggacgagttgcctcctcaaagggactgacggtttgcagactatcagagaacatgttccttctgtgatcagccacctaggctccgctaatgcgttccagcttccaggagaccctgcagaccccacctcccatgctctctgccctttacccctgtgctttgtacacactaggcacctgctctaccactgaacctcacacctacacctccatttttattttatttttgacacagtgtcttaaggtagtctggctgatgtctggctatcactcagcgctttttttgtatatcaatatttcattctttttcattgccaagtggtcttgtaaagacataccacagtgtgtcatccattcccagtgggtggatgggtgaatagctgcattgttttcagcttttgtgtacaccaataaaattgctgtaggtgttcagaaaaaaaaaaaaaaaa

SEQ ID NO: 4 - protein, 618 aa, Mus musculus
MEGAEAGARATFGPWDYGVFATMLLVSTGIGLWVGLARGGQRSADDFFTGGRQLAAVPVGLSLAASFMSAVQVLGVPAEAARYGLKFLWMCVGQLLNSLLTALLFLPIFYRLGLTSTYQYLELRFSRAVRLCGTLQYLVATMLYTGIVIYAPALILNQVTGLDIWASLLSTGIICTLYTTVGGMKAVVWTDVFQVVVMLVGFWVILARGVMLMGGPWNVLSLAQNHSRINLMDFDPDPRSRYTFWTFVVGGSLVWLSMYGVNQAQVQRYVACHTERKAKLALLVNQLGLFLIVASAACCGIVMFVYYKDCDPLLTGRIAAPDQYMPLLVLDIFEDLPGVPGLFLACAYSGTLSTASTSINAMAAVTVEDLIKPRMPSLAPRKLVFISKGLSFIYGSTCLTVAALSSLLGGGVLQGSFTVMGVISGPLLGAFTLGMLLPACNTPGVLSGLTAGLAVSLWVAVGATLYPPGEQTMGVLPTSAAGCTNASVLPSPPGAANTSRGIPSSGMDSGRPAFADTFYAVSYLYYGALGTLTTMLCGALISYLTGPTKRSSLGPGLLWWDLARQTASVAPKEDTTTLEDSLVKGPEDIPAATKKPPGFRPEAETHPLYLGHDAETNL

SEQ ID NO: 5 - DNA, 2862 bp, Rattus norvegicus
gcggtgactcgcgctgcgactctcccactgaccgagagtccccgacgtcctccgcatcctctcctcaccgagtcacctgtctccatggagggtgcggaggccggggcccgggccaccttcggcgcctgggactacggcgtgttcgcgaccatgctgctggtgtccacgggcatcgggctatgggtcggcctggcccgcggtggccaacgcagtgccgacgacttctttaccgggggccggcagttggcagccgttcctgtggggctgtcgctggccgccagtttcatgtcggctgtgcaggtgctcggggtccccgccgaggcagcgcgctacgggctcaagttcctgtggatgtgcgcgggtcagttgctcaactcgctgctcacagcgtttctcttcttgccgatcttctaccgcctgggccttaccagcacctaccagtacctagagctgcgcttcagccgagcggtccggctctgcgggacgctgcagtacttggtggccacgatgctgtatacaggcatcgtgatctacgcgcctgcgctcatcctgaaccaagtgaccgggttggacatctgggcatcgctcctgtccacaggaatcatctgcaccttgtacactaccgtgggtggtatgaaggccgtggtctggacagatgtgttccaggttgtggtaatgctcgttggcttctgggtgatcctggcccgaggcgtcattctcctggggggtccccggaacatgctcagcctcgctcagaaccattcccggatcaacctgatggactttgaccctgatcctcggagccggtacaccttctggactttcatagtgggtggcacactggtgtggctctccatgtacggtgtgaaccaagcccaggtacagcgctatgtggcctgccacacagagggaaaggccaaactggccctgcttgtcaaccagctgggcctcttcctgattgtggccagtgcagcttgctgtggcattgtcatgttcgtctactacaaggactgtgaccccctcctcacaggccgtatctcagcccccgaccagtacatgccgctgcttgtgttggacatttttgaggatctgcccggagtccccgggctcttcctggcctgtgcctacagtggcaccctcagcactgcatccaccagcatcaacgccatggcagctgtgactgtggaagacctcatcaagccgaggatgcctggcctggcacctcggaagttggttttcatctctaaagggctctcattcatctacggctctgcctgcctcactgtggctgctctgtcctcactgctgggaggtggtgtcctccagggttccttcactgtgatgggtgtcatcagtgggcctctactaggcgccttcacgcttgggatgctgctcccagcctgcaacacgccaggcgttctctccgggttggcagcaggcttggctgtatccctgtgggtggccgtaggggccacactgtatccccctggagagcagaccatgggggtgctgcccacctcggctgcaggctgcaccaacgattcggtcctcctgggcccacctggagccaccaacgcttccaacgggatccccagttctggaatggacacgggccgccctgccctcgctgataccttttacgccatctcctatctctattacggggctctgggcacgctgaccaccatgctttgcggtgctctcatcagctaccttactggtcccaccaagcgcagctccctgggtcccggattgctgtggtgggaccttgctcgacagacagcgtctgtggccccaaaggaagacactgccaccctggaggagagcctggtgaagggaccggaagacatccctgctgtgaccaagaagccccctggcctcaagccaggcgccgagacccaccccctgtatctggggcacgatgtggagaccaacctctgagggtggggtccaagaaggccaatcacaggcctcgggccagcagcctcctctctggatggttggacctgagcatatatagaagcttggctgatacatgccctgcccagaagtccctgtgtcttacccgcaccaaagagagagagagagcgagagagagagagagagagacagagagagagagagagagagagagacagagagagagagagagagagagagagagagagagaggagttggttctccatccacaaaggaaaccgtctggaaccttcatgcccttgtagatttcagtaggcagcggagaacactcagcttctccagactgaggttttctcatttatcaggcagagaaaccgagggctgtcaccccaacaccggggaggagacagtagaagggtcatagatacaaagaaaactaaggcagagggagaaatgaattgtctacagagcacagagctccaaggattgtgaagctaccttgaggtgccaagggacggattctcagagccttcacaagacacaaacggacgagttgcctcctccagttcagatggtttgcagactatcagagaacatgtttctcctgtgatcagctacctagcctctgccaacgtgttccagcttccaggaggccacacagaccccaccccccatgctctcaccctttacccctgtgcttttcacacactaggcaactgctccaccacaggacctcacacctagacctccgtttttgacacagggccttaaggtagtctggctgccatctgactatctctcagcacgttcacgtgtataatatttcattctttttcattgccaagttgtcttgtaaggagagaccacaatgtgtcatccatgcccagcttttgtgtctaacaaataaaatcgctgaaggtgttcaaaaaaaaaaaaaaaaa

SEQ ID NO: 6 - protein, 618 aa, Rattus norvegicus
MEGAEAGARATFGAWDYGVFATMLLVSTGIGLWVGLARGGQRSADDFFTGGRQLAAVPVGLSLAASFMSAVQVLGVPAEAARYGLKFLWMCAGQLLNSLLTAFLFLPIFYRLGLTSTYQYLELRFSRAVRLCGTLQYLVATMLYTGIVIYAPALILNQVTGLDIWASLLSTGIICTLYTTVGGMKAVVWTDVFQVVVMLVGFWVILARGVILLGGPRNMLSLAQNHSRINLMDFDPDPRSRYTFWTFIVGGTLVWLSMYGVNQAQVQRYVACHTEGKAKLALLVNQLGLFLIVASAACCGIVMFVYYKDCDPLLTGRISAPDQYMPLLVLDIFEDLPGVPGLFLACAYSGTLSTASTSINAMAAVTVEDLIKPRMPGLAPRKLVFISKGLSFIYGSACLTVAALSSLLGGGVLQGSFTVMGVISGPLLGAFTLGMLLPACNTPGVLSGLAAGLAVSLWVAVGATLYPPGEQTMGVLPTSAAGCTNDSVLLGPPGATNASNGIPSSGMDTGRPALADTFYAISYLYYGALGTLTTMLCGALISYLTGPTKRSSLGPGLLWWDLARQTASVAPKEDTATLEESLVKGPEDIPAVTKKPPGLKPGAETHPLYLGHDVETNL

(19) United States
(12) Patent Application Publication
(10) Pub. No.: US 2016/0228577 A1
(43) Pub. Date: Aug. 11, 2016

(54) TRANSGENIC ANIMALS FOR IN VIVO IMAGING

(71) Applicant: Biogen MA Inc., Cambridge, MA (US)

(72) Inventors: Ajay Verma, Needham Heights, MA (US); John William Hoppin, Boston, MA (US); Roger Askew, Lincoln, MA (US)

(73) Assignee: Biogen MA Inc., Cambridge, MA (US)

(21) Appl. No.: 15/029,443

(22) PCT Filed: Oct. 15, 2014
(86) PCT No.: PCT/US2014/060669

§ 371 Date: Apr. 14, 2016

Related U.S. Application Data
(60) Provisional application No. 61/891,129, filed on Oct. 15, 2013.

(51) Int. Cl. A61K 49/00; A01K 67/027

(52) U.S. Cl. CPC A61K 49/0008; A61K 49/0004; A01K 67/0278; A01K 2267/0393; A01K 2227/105; A01K 2217/052

(57) ABSTRACT

Compositions, methods and transgenic animals for in vivo detection of ectopically expressed sodium iodide symporter (NIS) are provided.

### CROSS-REFERENCE TO RELATED APPLICATIONS

This application claims the benefit of U.S. Provisional Application No. 61/891,129, filed Oct. 15, 2013, the entire contents of which are incorporated herein by reference.

### SEQUENCE LISTING

The instant application contains a Sequence Listing which has been submitted electronically in ASCII format and is hereby incorporated by reference in its entirety. Said ASCII copy, created on Oct. 13, 2014, is named B2047-7098WO_SL.txt and is 28,950 bytes in size.

### BACKGROUND

The use of reporter genes encoding proteins such as green-fluorescent protein (GFP), other fluorescent proteins, or luminescence generating enzymes such as luciferase has been useful in monitoring the in vivo expression pattern of transgenes. By directing the stable expression of such reporter genes into specific cells such as astrocytes, microglia, immune cells, specific neuronal pathways, and several organs via use of cell-specific promoters, investigators have been able to generate animals in which the biology of such cells can be studied under certain in vivo conditions. However, these approaches are limited by the relatively insensitive in vivo fluorescent or luminescent imaging approaches they rely upon, which are not ideally suited for macroscopic imaging. The lack of resolution of whole body fluorescent and luminescent imaging approaches has limited the capabilities of reporter transgene approaches since it is currently not possible to accurately localize whole body fluorescent and luminescent signals to specific tissues.

To gain this information, investigators are currently required to further examine excised tissue ex vivo or under in vivo surgical preparations for visual and microscopic access to tissue. These surgical and terminal animal experiments carry the risk of artificially altering transgene expression and also markedly increase the expense of transgenic animal experiments due to the need for animal sacrifice.

Reporter systems have been developed that allow high resolution imaging using magnetic resonance imaging (MRI). These reporter systems require the expression of molecules that mediate tissue uptake of MRI sensitive metals such as gadolinium and iron oxides (Geraldes CFGC, Laurent S, Classification and basic properties of contrast agents for magnetic resonance imaging, Contrast Media & Mol Imaging, 4:1-23, 2009). Although MRI imaging approaches allow resolution in the 100-500 μm range, the detection range for MRI sensitive molecules is in the micromolar to millimolar range. This low sensitivity is not ideal for studying in vivo processes since many of the tracer molecules are not accumulated by tissues in sufficiently high concentrations and can also be toxic at these concentrations.

Thus, the need exists for improved methods to detect gene expression and assess cells and/or tissues in vivo.

### SUMMARY OF THE INVENTION

The present disclosure provides, at least in part, compositions, methods and transgenic animals (e.g., transgenic non-human animals) for in vivo detection of ectopically expressed sodium iodide symporter (NIS).

Thus, in one aspect, the present disclosure features a transgenic animal having somatic and germ cells comprising a stably integrated nucleic acid for ectopic protein expression in transgenic animal cells of a sequence encoding a sodium iodide symporter (NIS) protein or biologically active fragment thereof. In some embodiments, the animal is selected from a mouse, rat or rabbit. In some embodiments, the animal is a mouse. In certain embodiments, the nucleic acid is stably integrated by targeted integration. In certain embodiments, the nucleic acid is stably integrated by random integration.

In particular embodiments, the stably integrated nucleic acid includes a promoter region that is operably linked to the sequence encoding the NIS protein or biologically active fragment thereof. In some embodiments, the promoter region is selected from a human promoter region, a mouse promoter region, a rat promoter region, or a viral promoter region. In some embodiments, the promoter is a constitutive promoter, e.g., thy-1. In some embodiments, the promoter comprises an inducible promoter element, e.g., hypoxia responsive element (HRE), antioxidant response element (ARE), proteolipid protein (PLP) promoter, glial fibrillary acidic protein (GFAP) promoter, Stop-FLox element.

In some embodiments, the nucleic acid is stably integrated into the animal cells at a genomic locus such that an endogenous promoter is operably linked to the sequence encoding the NIS protein or biologically active fragment thereof. In some embodiments, the stably integrated nucleic acid is integrated at a genomic locus that is 3′ to the endogenous promoter.

In certain embodiments, the NIS protein or biologically active fragment thereof is selected from a human NIS protein, e.g., SEQ ID NO:2, or biologically active fragment thereof, a mouse NIS protein, e.g., SEQ ID NO:4, or biologically active fragment thereof, or a rat NIS protein, e.g., SEQ ID NO:6, or biologically active fragment thereof. In some embodiments, the nucleic acid comprises a nucleic acid selected from SEQ ID NO:1, SEQ ID NO:3, or SEQ ID NO:5.

In some embodiments, the stably integrated nucleic acid further comprises one or more reporter genes, e.g., fluorescent protein, e.g., GFP, e.g., luminescence generating enzyme, e.g., luciferase. In particular embodiments, the one or more reporter genes is under control of the same promoter region that directs expression of the NIS protein. In particular embodiments, the one or more reporter genes is under control of a different promoter region that directs expression of the NIS protein.

In certain embodiments, the nucleic acid comprises a peptide cleavage element, e.g., an autocatalytic peptide cleavage element, e.g., P2A, T2A.

In some embodiments, iodine or technetium uptake is increased relative to a wild-type animal in one or more tissues, e.g., tissues that are not thyroid gland epithelium, gastric epithelium, nasolacrimal duct epithelium, or lactating mammary gland epithelium.

In embodiments, a NIS protein, or biologically active fragment thereof, is expressed, and wherein the transgenic animal is fertile and passes to its offspring the nucleic acid encoding the NIS protein, or biologically active fragment thereof. In embodiments, the transgenic animal is fertile and passes to its offspring the nucleic acid comprising a nucleic acid selected from SEQ ID NO:1, SEQ ID NO:3, or SEQ ID NO:5.

In some embodiments, the animal is hemizygous or heterozygous for the stably integrated nucleic acid, e.g., a founder animal. In some embodiments, the animal is homozygous for the stably integrated nucleic acid.

In another aspect, the present disclosure features an isolated cell from the transgenic animals disclosed herein. In some embodiments, the isolated cell is a somatic cell. In some embodiments, the isolated cell is a germ cell. In particular embodiments, the cell ectopically expresses the NIS protein or biologically active fragment thereof.

In yet another aspect, the present disclosure features a founder transgenic animal that is hemizygous for somatic and germ cells comprising a stably integrated nucleic acid for ectopic protein expression in transgenic animal cells of a sequence encoding a sodium iodide symporter (NIS) protein or biologically active fragment thereof.

In a further aspect, the present disclosure features a method of making a transgenic animal comprising crossing two founder animals provided herein. In another aspect, the present disclosure features a progeny animal resulting from such a cross.

In a further aspect, the present disclosure features an in vivo method of detecting NIS activity in a transgenic animal, comprising: administering to a transgenic animal of any of the preceding claims a detectable substrate of NIS; and detecting the detectable substrate in the animal. In some embodiments, the NIS activity is ion transport e.g., anion uptake into cells, cation export from cells. In particular embodiments, the detectable substrate is a detectable anion, e.g., radiolabeled iodine or technetium anion. In certain embodiments, the detecting comprises imaging or visualizing the detectable substrate in the animal. In some embodiments, the substrate is detected by single photon emission computed tomography (SPECT), Positron Emission Tomography (PET), Magnetic Resonance Imaging (MRI) or scintigraphy.

In particular embodiments, the animal is living at the time of detecting the substrate of NIS. In some embodiments, the administering and detecting are performed at more than one timepoint, e.g., 2, 3, 4, 5, 6, 7, 8, 9, 10 or more timepoints, in the same animal. In certain embodiments, the substrate of NIS is detected at more than one timepoint, e.g., 2, 3, 4, 5, 6, 7, 8, 9, 10 or more timepoints, after administration of the detectable substrate in the same animal.

In some embodiments, the resolution of the substrate detection is high resolution, e.g., about 0.5 to about 2 mm. In some embodiments, the sensitivity of the substrate detection is high sensitivity, e.g., picomolar to nanomolar detection sensitivity.

In some embodiments, the method further includes administering an agent, e.g., a pharmacologic agent, to the transgenic animal, e.g., before, after or simultaneously with the detectable substrate, e.g., before, after or simultaneously with the detecting of the substrate.

In another aspect, the present invention provides a method of generating an NIS transgenic animal model disease model, the method comprising: crossing the transgenic animal disclosed herein with an animal of the same species having a genetic predisposition for development of a disease, disorder or condition, thereby generating one or more progeny. In certain embodiments, the transgenic animal is selected from a mouse, rat or rabbit. In some embodiments, the disease, disorder or condition is selected from cancer, heart disease, hypertension, metabolic and hormonal disorders, diabetes, obesity, osteoporosis, glaucoma, skin pigmentation diseases, blindness, deafness, neurodegenerative disorders (e.g., CNS demyelinating diseases, CNS injury, Amyotrophic lateral sclerosis (ALS), Huntington's disease, Alzheimer's disease, Parkinson's disease, diabetic neuropathy, stroke, idiopathic inflammatory demyelinating disease, multiple sclerosis (MS), optic neuritis (e.g., acute optic neuritis), neuromyelitis optica (NMO), leukodystrophies, vitamin B12 deficiency, progressive multifocal leukoencephalopathy (PML), encephalomyelitis (EPL), acute disseminated encephalomyelitis (ADEM), central pontine myelolysis (CPM), Wallerian Degeneration, adrenoleukodystrophy, Alexander's disease, Pelizaeus Merzbacher disease (PMZ), traumatic glaucoma, periventricular leukomalatia (PVL), or transverse myelitis), psychiatric disturbances (e.g., anxiety or depression), or birth defects (e.g., cleft palate or anencephaly). In a further aspect, the present disclosure features a progeny animal resulting from such a cross.

In yet another aspect, the present disclosure features a method of generating an NIS transgenic animal model disease model, the method comprising: administering to the transgenic animal provided herein a compound or a treatment to induce disease; administering to the animal a detectable substrate of NIS; and detecting the detectable substrate in the animal at one or more predetermined time intervals. In some embodiments, the compound is a pharmaceutical compound. In particular embodiments, the treatment is exposure to a disease inducing agent.

In some embodiments, the detectable substrate is a detectable anion, such as radiolabeled iodine or technetium anion. In certain embodiments, the detecting comprises imaging or visualizing the detectable substrate in the animal.

In another aspect, the present disclosure features a method of evaluating a candidate disease therapy comprising: administering to an animal provided herein a test compound; administering to the animal a detectable substrate of NIS; and detecting a change in the detectable substrate in the animal at one or more predetermined time intervals; thereby identifying a test compound as a candidate disease therapy.

In yet a further aspect, the present disclosure features a method of monitoring disease progression, comprising: obtaining a disease model animal by the method provided herein; administering to the animal a detectable substrate of NIS; and detecting the detectable substrate in the animal at one or more predetermined time intervals; thereby monitoring disease progression in the animal.

In some embodiments, the detectable substrate is a detectable anion, such as radiolabeled iodine or technetium anion. In certain embodiments, the detecting comprises imaging or visualizing the detectable substrate in the animal. In particular embodiments, the substrate is detected by single photon emission computed tomography (SPECT), Positron Emission Tomography (PET), Magnetic Resonance Imaging (MRI) or scintigraphy.

In certain embodiments, the animal is living at the time of detecting the substrate of NIS. In some embodiments, the administering and detecting are performed at more than one timepoint, e.g., 2, 3, 4, 5, 6, 7, 8, 9, 10 or more timepoints, in the same animal. In some embodiments, the substrate of NIS is detected at more than one timepoint, e.g., 2, 3, 4, 5, 6, 7, 8, 9, 10 or more timepoints, after administration of the detectable substrate in the same animal. In some embodiments, the test compound is administered at more than one timepoint, e.g., 2, 3, 4, 5, 6, 7, 8, 9, 10 or more timepoints.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 depicts exemplary stable transgenic NIS genetic constructs for (a) random integration into the genome; (b) integration of an inducible transgene either randomly or into a predetermined genomic locus; and (c) gene targeted integration of the NIS reporter to a gene of interest from an endogenous promoter.

FIG. 2 depicts the design of an exemplary construct for generation of NIS transgenic mouse line.

FIG. 3 depicts the results of qPCR genotyping for F₀ pups using a GFP probe.

### DETAILED DESCRIPTION

The invention is based, at least in part, on the discovery that transgenic animals ectopically expressing sodium iodide symporter (NIS) are valuable for, inter alia, monitoring, e.g., the integrity, health, behavior and gene expression regulation of various cells and tissues in vivo. Furthermore, provided transgenic animals ectopically expressing NIS may be imaged using various in vivo imaging techniques, such as SPECT and PET imaging, which provide high sensitivity and high resolution imaging. Provided are methods, compositions and transgenic animals for in vivo NIS activity detection.

### DEFINITIONS

For convenience, before further description of the present invention, certain terms employed in the specification, examples and appended claims are defined here. Other terms are defined as they appear in the specification.

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a physical process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non covalent bond, between a first and a second atom of the analyte; or by changing the structure of a reagent, or a fragment or other derivative thereof, e.g., by breaking or forming a covalent or non covalent bond, between a first and a second atom of the reagent.

“Acquiring a sample” as the term is used herein, refers to obtaining possession of a sample, e.g., a tissue sample or nucleic acid sample, by “directly acquiring” or “indirectly acquiring” the sample. “Directly acquiring a sample” means performing a process (e.g., performing a physical method such as a surgery or extraction) to obtain the sample. “Indirectly acquiring a sample” refers to receiving the sample from another party or source (e.g., a third party laboratory that directly acquired the sample). Directly acquiring a sample includes performing a process that includes a physical change in a physical substance, e.g., a starting material, such as a tissue, e.g., a tissue in a human patient or a tissue that has was previously isolated from a patient. Exemplary changes include making a physical entity from a starting material, dissecting or scraping a tissue; separating or purifying a substance (e.g., a sample tissue or a nucleic acid sample); combining two or more separate entities into a mixture; performing a chemical reaction that includes breaking or forming a covalent or non-covalent bond. Directly acquiring a sample includes performing a process that includes a physical change in a sample or another substance, e.g., as described above.

The term “animal” is used herein to include all vertebrate animals, except humans (e.g., primates, equines, canines, felines, rodents, ovines, bovines, avians, and the like). It also includes an individual animal in all stages of development, including embryonic and fetal stages.

“Biologically active” refers to those forms of proteins and polypeptides, including conservatively substituted variants, alleles of genes encoding a protein or polypeptide fragments of proteins which retain a biological and/or immunological activity of the wild-type protein or polypeptide. For example, a biologically active form of sodium iodide symporter (NIS) protein or polypeptide is preferably one which induces transport of anions, e.g., iodide, into a cell and transport of cations, e.g., sodium, out of a cell, whether directly or indirectly and whether in vivo or in an in vitro assay.

As used herein, the term “cDNA” refers to complementary or copy DNA produced from an RNA template by the action of RNA-dependent DNA polymerase (reverse transcriptase). Thus, a “cDNA clone” means a duplex DNA sequence for which one strand is complementary to an RNA molecule of interest, carried in a cloning vector or PCR amplified. cDNA can also be single stranded after first strand synthesis by reverse transcriptase. In this form it is a useful PCR template and does not need to be carried in a cloning vector. This term includes genes from which the intervening sequences have been removed. Thus, the term “gene”, as sometimes used generically, can also include nucleic acid molecules comprising cDNA and cDNA clones.

As used herein, the term “cloning vehicle” refers to a plasmid or phage DNA or other DNA sequence which is able to replicate in a host cell. This term can also include artificial chromosomes such as BACs and YACs. The cloning vehicle is characterized by one or more endonuclease recognition sites at which such DNA sequences may be cut in a determinable fashion without loss of an essential biological function of the DNA, which may contain a marker suitable for use in the identification of transformed cells.

As used herein, the term “expression” refers to the process comprising transcription of a gene sequence and subsequent processing steps, such as translation of a resultant mRNA to produce the final end product of a gene. The end product may be a protein (such as an enzyme or receptor) or a nucleic acid (such as a tRNA, antisense RNA, or other regulatory factor). The term “expression control sequence” refers to a sequence of nucleotides that control or regulate expression of structural genes when operably linked to those genes. These include, for example, the lac systems, the trp system, major operator and promoter regions of the phage lambda, the control region of fd coat protein and other sequences known to control the expression of genes in prokaryotic or eukaryotic cells. Expression control sequences will vary depending on whether the vector is designed to express the operably linked gene in a prokaryotic or eukaryotic host, and may contain transcriptional elements such as enhancer elements, termination sequences, tissue-specificity elements and/or translational initiation and termination sites.

As used herein, the term “expression vehicle” refers to a vehicle or vector similar to a cloning vehicle but which is capable of expressing a gene which has been cloned into it, after transformation into a host. The cloned gene is usually placed under the control of (e.g., operably linked to) an expression control sequence.

Use used herein, a “fragment” of a gene refers to any portion of a gene sequence. A biologically active fragment refers to any portion of the gene that retains at least one biological activity of that gene.

As used herein, the term “gene” refers to a DNA sequence that encodes through its template or messenger RNA a sequence of amino acids characteristic of a specific peptide. The term “gene” includes intervening, non-coding regions, as well as regulatory regions, and can include 5′ and 3′ ends.

As used herein, the term “gene sequence” refers to a nucleic acid molecule, including DNA which contains a non-transcribed or non-translated sequence, which comprises a gene. The term is also intended to include any combination of gene(s), gene fragment(s), non-transcribed sequence(s) or non-translated sequence(s) which are present on the same DNA molecule.

As used herein, the term “host” includes prokaryotes and eukaryotes, such as yeast and filamentous fungi, as well as plant and animal cells. The term includes an organism or cell that is the recipient of a replicable expression vehicle.

As used herein, the term “isolated” refers to a substance altered by hand of man from the natural environment. An isolated peptide may be for example in a substantially pure form or otherwise displaced from its native environment such as by expression in an isolated cell line or transgenic animal. An isolated sequence may for example be a molecule in substantially pure form or displaced from its native environment such that at least one end of said isolated sequence is not contiguous with the sequence it would be contiguous with in nature.

As used herein, the term “operator” refers to a DNA sequence capable of interacting with the specific repressor, thereby controlling the transcription of adjacent gene(s).

As used herein, the term “operably linked” generally means that the promoter controls the initiation of expression of the gene. A promoter is operably linked to a sequence of proximal DNA if upon introduction into a host cell the promoter determines the transcription of the proximal DNA sequence(s) into one or more species of RNA. A promoter is operably linked to a DNA sequence if the promoter is capable of initiating transcription of that DNA sequence.

As used herein, the term “promoter” refers to a DNA sequence that can be recognized by an RNA polymerase. The presence of such a sequence permits the RNA polymerase to bind and initiate transcription of operably linked gene sequences. “Promoter region” is intended to include the promoter as well as other gene sequences which may be necessary for the initiation of transcription. The presence of a promoter region is sufficient to cause the expression of an operably linked gene sequence. The term “promoter” is sometimes used in the art to generically indicate a promoter region. Many different promoters are known in the art which direct expression of a gene in a certain cell types. Tissue-specific promoters can comprise nucleic acid sequences which cause a greater (or decreased) level of expression in cells of a certain tissue type. In some embodiments, a promoter is an endogenous promoter. In some embodiments, a promoter is an exogenous promoter.

As used herein, the term “probe” refers to any molecule which is capable of selectively binding to a specifically intended target molecule, for example, a transcription product, e.g., an mRNA or cDNA, or a translation product, e.g., a polypeptide or protein. Probes can be either synthesized by one skilled in the art, or derived from appropriate biological preparations. For purposes of detection of the target molecule, probes can be specifically designed to be labeled, as described herein. Examples of molecules that can be utilized as probes include, but are not limited to, RNA, DNA, proteins, antibodies, and organic monomers.

As used herein, the term “recombinant DNA” refers to a molecule that has been engineered by splicing in vitro a cDNA or genomic DNA sequence or altering a sequence by methods such as PCR mutagenesis.

“Sample,” “tissue sample,” “subject or patient sample,” “subject or patient cell or tissue sample” or “specimen” each refers to a biological sample obtained from a tissue, e.g., a bodily fluid, of a subject or patient. The source of the tissue sample can be solid tissue as from a fresh, frozen and/or preserved organ, tissue sample, biopsy, or aspirate; blood or any blood constituents (e.g., serum, plasma); bodily fluids such as cerebral spinal fluid, whole blood, plasma and serum. The sample can include a non-cellular fraction (e.g., plasma, serum, or other non-cellular body fluid). In one embodiment, the sample is a serum sample. In other embodiments, the body fluid from which the sample is obtained from an individual comprises blood (e.g., whole blood). In certain embodiments, the blood can be further processed to obtain plasma or serum. In another embodiment, the sample contains a tissue, cells (e.g., peripheral blood mononuclear cells (PBMC)). For example, the sample can be a fine needle biopsy sample, an archival sample (e.g., an archived sample with a known diagnosis and/or treatment history), a histological section (e.g., a frozen or formalin-fixed section, e.g., after long term storage), among others. The term sample includes any material obtained and/or derived from a biological sample, including a polypeptide, and nucleic acid (e.g., genomic DNA, cDNA, RNA) purified or processed from the sample. Purification and/or processing of the sample can involve one or more of extraction, concentration, antibody isolation, sorting, concentration, fixation, addition of reagents and the like. The sample can contain compounds that are not naturally intermixed with the tissue in nature such as preservatives, anticoagulants, buffers, fixatives, nutrients, antibiotics or the like.

As used herein, the term “selectable marker” refers to a gene which encodes, e.g., an enzymatic activity that confers resistance to an antibiotic or drug upon the cell in which the selectable marker is expressed. Selectable markers may be “positive”; positive selectable markers are typically dominant selectable markers, e.g., genes which encode an enzymatic activity which can be detected whenever present in a cell or cell line (including ES cells). Exemplary “positive” selectable markers include the bacterial aminoglycoside 3′ phosphotransferase gene (‘neo’) which confers resistance to the drug G418 in mammalian cells, the bacterial hygromycin G phosphotransferase gene (hyg) which confers resistance to the antibiotic hygromycin, the bacterial xanthine-guanine phosphoribosyl transferase gene (also referred to as the gpt gene) which confers the ability to grow in the presence of mycophenolic acid, as well as the hprt gene, the nDtll gene, or other genes which confer resistance to amino acid or nucleoside analogues; or antibiotics, among others. DNA encoding the positive selectable marker in the transgene (e.g., neo^{R}) is generally linked to an expression regulation sequence that allows for its independent transcription in ES cells. Moreover, “negative” selectable markers may also be used, which encode an enzymatic activity whose expression is cytotoxic to the cell when grown in an appropriate selective medium. For example, the HSV-tk gene is used as a negative selectable marker. Expression of the HSV-tk gene in cells grown in the presence of gancyclovir or acyclovir is cytotoxic; thus growth of cells in selective medium containing gancyclovir or acyclovir selects against cells capable of expressing a functional HSV-TK enzyme.

The term “target tissue,” as used herein generally refers to any tissue in the body of any subject, including the human body that comprises all the organs, structures and other contents. Specifically, a tissue is any substance made up of cells that perform a similar function within an organism. For example, tissue may refer to any epithelial tissue, connective tissue, muscle tissue, such as cardiac muscle, smooth muscle, and skeletal muscle, and any nervous tissue, such as tissue within the brain, spinal cord, and/or peripheral nervous system.

The term “test compound” refers to an agent that is to be screened in one or more of the assays described herein. The agent can be virtually any compound, e.g., chemical compound. It can exist as a single isolated compound or can be a member of a chemical (e.g., combinatorial) library. In a some embodiments, the test compound is a small molecule.

The term “transgene” as used herein refers to a foreign nucleic acid sequence that is placed into a subject animal, e.g., by introducing the foreign sequence into embryonic stem (ES) cells, newly fertilized eggs or early embryos. The term “foreign nucleic acid sequence” refers to any nucleic acid sequence which is introduced into the genome of an animal by experimental manipulations and may include nucleic acid sequences found in that animal so long as the introduced gene contains some modification (e.g., an immunoreactive epitope tag, a point mutation, the presence of a selectable marker gene, the presence of a loxP site, etc.) relative to the naturally-occurring gene.

A “transgenic animal” is an animal containing one or more cells bearing genetic information received, directly or indirectly, by deliberate genetic manipulation or by inheritance from a manipulated progenitor at a subcellular level, such as by microinjection or infection with a recombinant viral vector (e.g., adenovirus, retrovirus, herpes virus, adeno-associated virus, lentivirus). This introduced DNA molecule may be integrated within a chromosome, or it may be extra-chromosomally replicating DNA. In some embodiments, the introduced DNA molecule encodes an NIS protein or biologically active fragment thereof. In some embodiments, the introduced DNA molecule directs expression of a protein, e.g., an NIS protein or biologically active fragment thereof, in cells or tissue that do not naturally express the protein, or at higher levels of expression that is naturally present in the cells or tissue. A transgenic animal may be hemizygous, heterozygous, or homozygous for the introduced DNA molecule.

A disease or disorder is “treated,” “inhibited” or “reduced,” if at least one symptom of the disease is reduced, alleviated, terminated, slowed, or prevented.

As used herein, the term “variant” refers to a gene that is substantially similar in structure and biological activity or immunological characteristics to either the entire gene or to a fragment of the gene. Provided that the two genes possess a similar activity, they are considered variant as that term is used herein even if the sequence of encoded amino acid residues is not identical.

Headings, including alphabetical or numerical headings, are merely for ease of understanding and reading and, absent express indication to the contrary, do not impose temporal order of a hierarchy of preferences.

Various aspects of the invention are described in further detail below. Additional definitions are set out throughout the specification.

### Sodium Iodide Symporter (NIS)

The Sodium Iodide Symporter (NIS), which is also called the sodium/iodide cotransporter or solute carrier family 5, member 5 (SLC5A5), is a transmembrane glycoprotein that transports two sodium cations for each iodide anion into the cell. In humans, the NIS has a molecular weight of 87 kDa and has 13 transmembrane domains. NIS is normally only expressed in a limited set of tissues, such as the thyroid gland epithelium, gastric epithelium, nasolacrimal duct epithelium, and lactating mammary gland epithelium (Dohan O, De La Viega A, Paroder V, Riedel C, Artani M, Reed M, Ginter C, Carrasco N, The Sodium/Iodide Symporter (NIS): Characterization, Regulation and Medical Significance, Endocrine Reviews 24(1):48-77, 2003). Ectopic expression of NIS in various tissues may be achieved, e.g., using methods known in the art and/or methods provided herein. Exemplary nucleic acid and protein sequences of NIS from human, mouse and rat are provided below in Tables 1, 2, and 3, respectively.

TABLE 1

| Acc. | Human: Homo sapiens solute carrier family 5 (sodium iodide |
|---|---|
| # | symporter), member 5 (SLC5A5) |
| NM_00 | 1 gctgtcagcg ctgagcacag cgcccaggga gagggacaga cagccggctg catgggacag |
| 0453 | 61 cggaacccag agtgagaggg gaggtggcag gacagacaga cagcaggggc ggacgcagag |
| | 121 acagacagcg gggacaggga ggccgacacg gacatcgaca gcccatagat tcctaaccca |
| | 181 gggagccccg gcccctctcg ccgcttccca ccccagacgg agcggggaca ggctgccgag |
| | 241 catcctccca cccgccctcc ccgtcctgcc tcctcggccc ctgccagctt cccccgcttg |
| | 301 agcacgcagg gcgtccgagg acgcgctggg cctccgcacc cgccctcatg gaggccgtgg |
| | 361 agaccgggga acggcccacc ttcggagcct gggactacgg ggtctttgcc ctcatgctcc |
| | 421 tggtgtccac tggcatcggg ctgtgggtcg ggctggctcg gggcgggcag cgcagcgctg |
| | 481 aggacttctt caccgggggc cggcgcctgg cggccctgcc cgtgggcctg tcgctgtctg |
| | 541 ccagcttcat gtcggccgtg caggtgctgg gcgtgccgtc ggaggcctat cgctatggcc |
| | 601 tcaagttcct ctggatgtgc ctgggccagc ttctgaactc ggtcctcacc gccctgctct |
| | 661 tcatgcccgt cttctaccgc ctgggcctca ccagcaccta cgagtacctg gagatgcgct |
| | 721 tcagccgcgc agtgcggctc tgcgggactt tgcagtacat tgtagccacg atgctgtaca |
| | 781 ccggcatcgt aatctacgca ccggccctca tcctgaacca agtgaccggg ctggacatct |
| | 841 gggcgtcgct cctgtccacc ggaattatct gcaccttcta cacggctgtg ggcggcatga |
| | 901 aggctgtggt ctggactgat gtgttccagg tcgtggtgat gctaagtggc ttctgggttg |
| | 961 tcctggcacg cggtgtcatg cttgtgggcg ggccccgcca ggtgctcacg ctggcccaga |
| | 1021 accactcccg gatcaacctc atggacttta accctgaccc gaggagccgc tatacattct |
| | 1081 ggacttttgt ggtgggtggc acgttggtgt ggctctccat gtatggcgtg aaccaggcgc |
| | 1141 aggtgcagcg ctacgtggct tgccgcacag agaagcaggc caagctggcc ctgctcatca |
| | 1201 accaggtcgg cctgttcctg atcgtgtcca gcgctgcctg ctgtggcatc gtcatgtttg |
| | 1261 tgttctacac tgactgcgac cctctcctcc tggggcgcat ctctgcccca gaccagtaca |
| | 1321 tgcctctgct ggtgctggac atcttcgaag atctgcctgg agtccccggg cttttcctgg |
| | 1381 cctgtgctta cagtggcacc ctcagcacag catccaccag catcaatgct atggctgcag |
| | 1441 tcactgtaga agacctcatc aaacctcggc tgcggagcct ggcacccagg aaactcgtga |
| | 1501 ttatctccaa ggggctctca ctcatctacg gatcggcctg tctcaccgtg gcagccctgt |
| | 1561 cctcactgct cggaggaggt gtccttcagg gctccttcac cgtcatggga gtcatcagcg |
| | 1621 gccccctgct gggagccttc atcttgggaa tgttcctgcc ggcctgcaac acaccgggcg |
| | 1681 tcctcgcggg actaggcgcg ggcttggcgc tgtcgctgtg ggtggccttg ggcgccacgc |
| | 1741 tgtacccacc cagcgagcag accatgaggg tcctgccatc gtcggctgcc cgctgcgtgg |
| | 1801 ctctctcagt caacgcctct ggcctcctgg acccggctct cctccctgct aacgactcca |
| | 1861 gcagggcccc cagctcagga atggacgcca gccgacccgc cttagctgac agcttctatg |
| | 1921 ccatctccta tctctattac ggtgccctgg gcacgctgac cactgtgctg tgcggagccc |
| | 1981 tcatcagctg cctgacaggc cccaccaagc gcagcaccct ggccccggga ttgttgtggt |
| | 2041 gggacctcgc acggcagaca gcatcagtgg cccccaagga agaagtggcc atcctggatg |
| | 2101 acaacttggt caagggtcct gaagaactcc ccactggaaa caagaagccc cctggcttcc |
| | 2161 tgcccaccaa tgaggatcgt ctgtttttct tggggcagaa ggagctggag ggggctggct |
| | 2221 cttggacccc ctgtgttgga catgatggtg gtcgagacca gcaggagaca aacctctgag |
| | 2281 gacagggcca gccgcgggac tgacaccctg ggatggaacc tcaggatggg ccaaacccag |
| | 2341 acaacgggcc catggccttg ggctctgatt ggctggattg ccttgtatgc aaatgagttc |
| | 2401 aggactacaa taccctaccc tatggggagg ccctgcctcc gggaggtcat tttttaaatc |
| | 2461 cagccccttg cttcaaccgt ccccagtatt agacgctgca gccctgacgg ctccccccaa |
| | 2521 ataaggctgg gtttttctct ctctcttttt tttttttttt tttttttgag acagggtcat |
| | 2581 gctctgtcac ccaggctgga gtgcagtggt gtgatctcgg ctcactgcaa cctctgcctc |
| | 2641 ccaggctcaa gtgattctcc tgcctcagcc tcttgagtag ctgggattgt aggtgcccac |
| | 2701 caccatgccc agccaacttt ttgtattttt agtagagaca gggtttcacc atgttggtca |
| | 2761 ggctggtctc gaactcctga cctcaagtga tccacctgcc tcggcctccc aaagtgctgg |
| | 2821 gttacaggcg taagctacca tgcccagcct accgtttttc tcaatctata atagaaagcc |
| | 2881 accacgccca ggtaattttt gtaattttgt atttttgtag agacggggtt ttgccatgtt |
| | 2941 ggccaggctg gtctcaaact cctgatctca ggtgatcctc ctgccttggc ctcccaaagg |
| | 3001 gctgggatta caggggtgag ccaccgcgcc ccacctcttt cttattttct tcctgggatt |
| | 3061 ggggagggga tgattcagac cccacatggc ctccaacctt ggcccacaca cctgccatgg |
| | 3121 ctcccatcat cctgagcatg ctagcgtccc ctcctcacct gacaatggag gctctcgaat |
| | 3181 tgggttgtgt ccccccaaaa tttatgtcta cccagaacct cagaacatga gcttacttgg |
| | 3241 aaatagggtc tttgcaggtg taactggtta aattaaaaga ggtattactg gaggaggatg |
| | 3301 gatgaatcca gtgactggtt cctcatatga agtagagaag agatgcagag aaacacatgg |
| | 3361 ggaagatgcc acgtgaagac agaggcagtg gttggatcaa tgcatctacg agtcggagaa |
| | 3421 cccaaggatt gccagcaaca accagaaatc aggagggggg catgagatgc attatttctt |
| | 3481 agagccttta gagggaacat ggccctactg acaccttgat gtcagacttc tggctgctag |
| | 3541 aactgtcaga gaataaattt ctgttgtttg atgccaaaaa aaaaaaaaaa aaaa |
| | (SEQ ID NO: 1) |
| NG_01 | MEAVETGERPTFGAWDYGVFALMLLVSTGIGLWVGLARGGQRSAEDFFTGGRRLAALPVGLSLSASFMSAVQV |
| 2930 | LGVPSEAYRYGLKFLWMCLGQLLNSVLTALLFMPVFYRLGLTSTYEYLEMRFSRAVRLCGTLQYIVATMLYTG |
| | IVIYAPALILNQVTGLDIWASLLSTGIICTFYTAVGGMKAVVWTDVFQVVVMLSGFWVVLARGVMLVGGPRQV |
| | LTLAQNHSRINLMDFNPDPRSRYTFWTFVVGGTLVWLSMYGVNQAQVQRYVACRTEKQAKLALLINQVGLFLI |
| | VSSAACCGIVMFVFYTDCDPLLLGRISAPDQYMPLLVLDIFEDLPGVPGLFLACAYSGTLSTASTSINAMAAV |
| | TVEDLIKPRLRSLAPRKLVIISKGLSLIYGSACLTVAALSSLLGGGVLQGSFTVMGVISGPLLGAFILGMFLP |
| | ACNTPGVLAGLGAGLALSLWVALGATLYPPSEQTMRVLPSSAARCVALSVNASGLLDPALLPANDSSRAPSSG |
| | MDASRPALADSFYAISYLYYGALGTLTTVLCGALISCLTGPTKRSTLAPGLLWWDLARQTASVAPKEEVAILD |
| | DNLVKGPEELPTGNKKPPGFLPTNEDRLFFLGQKELEGAGSWTPCVGHDGGRDQQETNL |
| | (SEQ ID NO: 2) |

TABLE 2

| Acc. | Mouse: Mus musculus solute carrier family 5 (sodium iodide |
|---|---|
| # | symporter), member 5 (SLC5A5) |
| NM_05 | 1 ggtcgacggc ccagcaggaa ctcgcgctgc gacgacgctc gctctgagag tccccgacgt |
| 3248 | 61 cctccgcatc ctctccgttc cgagttacct gtctccatgg agggcgcgga ggcaggggcc |
| | 121 cgggccacct tcggcccctg ggactacggc gtgttcgcga ccatgctgct ggtgtccacg |
| | 181 ggcatcggtc tgtgggtcgg cctggcccgc ggcggccagc gcagcgccga cgacttcttc |
| | 241 accgggggcc ggcagctggc ggcagtgcct gtggggctgt cgctggccgc tagcttcatg |
| | 301 tcggctgtgc aggtgctcgg ggtccccgcg gaggcagcgc gctacggtct caagtttctg |
| | 361 tggatgtgcg tgggccagtt gctcaattcg ctgctcacag ccttgctctt cttgccgatc |
| | 421 ttctaccgcc tgggccttac cagcacctac cagtacctag aactgcgctt cagccgagcg |
| | 481 gtccggctct gcgggacgct gcagtacttg gtggccacga tgctgtatac tggcatcgtg |
| | 541 atctacgcac ctgcgctcat cctgaaccaa gtgaccgggt tggacatctg ggcatcgctc |
| | 601 ctgtccacag gaatcatctg caccttgtac acgaccgtgg gtggtatgaa ggccgtggtc |
| | 661 tggacagatg tgttccaggt tgtggtaatg ctcgtcggct tctgggtgat cctggctcga |
| | 721 ggtgtcatgc tcatgggggg cccctggaac gtgctcagtc tcgctcaaaa ccattcccgg |
| | 781 atcaacctga tggactttga ccctgaccct cggagccgct acaccttctg gacatttgta |
| | 841 gtaggtggct ccctggtgtg gctctccatg tatggtgtga accaagccca ggtgcagcgt |
| | 901 tatgtggcct gtcacacgga gagaaaggcc aagcttgccc tgcttgtcaa ccagcttggc |
| | 961 ctcttcctga ttgtggctag tgcagcttgc tgtggaattg tcatgtttgt ctactacaag |
| | 1021 gactgcgacc ccctcctcac aggccgcatc gcagcccccg atcagtacat gccattgctc |
| | 1081 gtgttggaca tttttgagga tctgcccgga gtccccgggc tcttcctggc ctgtgcctac |
| | 1141 agtggcaccc tcagcaccgc gtccaccagt atcaacgcta tggcagctgt aactgtggaa |
| | 1201 gacctcatca agcccaggat gcctagcctg gcaccccgga agctggtttt catctctaaa |
| | 1261 gggctctcat tcatctatgg ctcaacctgc ctcactgtgg ctgctctgtc ctcgctgctg |
| | 1321 ggaggtgggg tcctccaggg ctctttcacc gtgatgggtg tcatcagtgg gcctctcctt |
| | 1381 ggcgctttca ccctcgggat gctgctccca gcctgcaaca cgccaggcgt cctctccggg |
| | 1441 ctgacagcag gcttagctgt atccctgtgg gtggccgtgg gggccacgct gtacccgcct |
| | 1501 ggagagcaga ccatgggggt gctgcccacc tcggctgcgg gatgcaccaa tgcctctgtc |
| | 1561 ctcccgagcc cacccggagc tgccaacact tccagaggga tccccagttc tggaatggac |
| | 1621 tcgggccgcc cagcctttgc cgacaccttt tatgctgtct cctatctcta ctacggggct |
| | 1681 ctgggtactt tgaccactat gctttgtggt gctctcatca gctacctaac tggccccacc |
| | 1741 aagcgcagct ccctgggccc cggattactg tggtgggacc tcgctcggca gacagcatct |
| | 1801 gtggccccaa aggaggacac caccaccctg gaggacagcc tggttaaggg accggaagac |
| | 1861 atccctgctg cgaccaagaa gccccctggc ttcaggccag aagccgagac ccaccccctg |
| | 1921 tatctggggc acgatgctga gaccaacctc tgagggcgag gcccgagaaa gccaatcaca |
| | 1981 ggcctcgggc cagcagcctc ctctctgaat ggttggacca tcacctgtac agaagcttgg |
| | 2041 ctgatagagg ccctgcccgc cctgaagtcc ctgtgtccca cctgtgcccc ccaaaagagg |
| | 2101 gttggttctc tatccaccaa ggaaaacatc tggaaccgca gtgaccttgt agattgcagt |
| | 2161 aggcaactga gaacactcag cttctccaga ccgtgaggtt ttcccattta acaagcagag |
| | 2221 aagctgaggg cggtcacccc aacgctggga aggtagaggc aagagattca ggaggtcaag |
| | 2281 atcatccaca gctacaggct gagaccctgc ccacccccaa atatataaaa gtaatctggg |
| | 2341 tgagccgcgt ggcttcgcga cagagggctt gcccagcaca cagaagaccc aaggcagagg |
| | 2401 gagaaatggg atgtccacag aacacagagc cccaaggatt gtgaagcttc cgcgaagtgc |
| | 2461 caagggacag attctcagag ccctcacaag acacggatgg acgagttgcc tcctcaaagg |
| | 2521 gactgacggt ttgcagacta tcagagaaca tgttccttct gtgatcagcc acctaggctc |
| | 2581 cgctaatgcg ttccagcttc caggagaccc tgcagacccc acctcccatg ctctctgccc |
| | 2641 tttacccctg tgctttgtac acactaggca cctgctctac cactgaacct cacacctaca |
| | 2701 cctccatttt tattttattt ttgacacagt gtcttaaggt agtctggctg atgtctggct |
| | 2761 atcactcagc gctttttttg tatatcaata tttcattctt tttcattgcc aagtggtctt |
| | 2821 gtaaagacat accacagtgt gtcatccatt cccagtgggt ggatgggtga atagctgcat |
| | 2881 tgttttcagc ttttgtgtac accaataaaa ttgctgtagg tgttcagaaa aaaaaaaaaa |
| | 2941 aaa (SEQ ID NO: 3) |
| NP | MEGAEAGARATFGPWDYGVFATMLLVSTGIGLWVGLARGGQRSADDFFTGGRQLAAVPVGLSLAASFMSAVQV |
| 44447 | LGVPAEAARYGLKFLWMCVGQLLNSLLTALLFLPIFYRLGLTSTYQYLELRFSRAVRLCGTLQYLVATMLYTG |
| 8.2 | IVIYAPALILNQVTGLDIWASLLSTGIICTLYTTVGGMKAVVWTDVFQVVVMLVGFWVILARGVMLMGGPWNV |
| | LSLAQNHSRINLMDFDPDPRSRYTFWTFVVGGSLVWLSMYGVNQAQVQRYVACHTERKAKLALLVNQLGLFLI |
| | VASAACCGIVMFVYYKDCDPLLTGRIAAPDQYMPLLVLDIFEDLPGVPGLFLACAYSGTLSTASTSINAMAAV |
| | TVEDLIKPRMPSLAPRKLVFISKGLSFIYGSTCLTVAALSSLLGGGVLQGSFTVMGVISGPLLGAFTLGMLLP |
| | ACNTPGVLSGLTAGLAVSLWVAVGATLYPPGEQTMGVLPTSAAGCTNASVLPSPPGAANTSRGIPSSGMDSGR |
| | PAFADTFYAVSYLYYGALGTLTTMLCGALISYLTGPTKRSSLGPGLLWWDLARQTASVAPKEDTTTLEDSLVK |
| | GPEDIPAATKKPPGFRPEAETHPLYLGHDAETNL (SEQ ID NO: 4) |

TABLE 3

| Acc. | Rat: Rattus norvegicus solute carrier family 5 (sodium iodide |
|---|---|
| # | symporter), member 5 (SLC5A5) |
| NM_05 | 1 gcggtgactc gcgctgcgac tctcccactg accgagagtc cccgacgtcc tccgcatcct |
| 2983 | 61 ctcctcaccg agtcacctgt ctccatggag ggtgcggagg ccggggcccg ggccaccttc |
| | 121 ggcgcctggg actacggcgt gttcgcgacc atgctgctgg tgtccacggg catcgggcta |
| | 181 tgggtcggcc tggcccgcgg tggccaacgc agtgccgacg acttctttac cgggggccgg |
| | 241 cagttggcag ccgttcctgt ggggctgtcg ctggccgcca gtttcatgtc ggctgtgcag |
| | 301 gtgctcgggg tccccgccga ggcagcgcgc tacgggctca agttcctgtg gatgtgcgcg |
| | 361 ggtcagttgc tcaactcgct gctcacagcg tttctcttct tgccgatctt ctaccgcctg |
| | 421 ggccttacca gcacctacca gtacctagag ctgcgcttca gccgagcggt ccggctctgc |
| | 481 gggacgctgc agtacttggt ggccacgatg ctgtatacag gcatcgtgat ctacgcgcct |
| | 541 gcgctcatcc tgaaccaagt gaccgggttg gacatctggg catcgctcct gtccacagga |
| | 601 atcatctgca ccttgtacac taccgtgggt ggtatgaagg ccgtggtctg gacagatgtg |
| | 661 ttccaggttg tggtaatgct cgttggcttc tgggtgatcc tggcccgagg cgtcattctc |
| | 721 ctggggggtc cccggaacat gctcagcctc gctcagaacc attcccggat caacctgatg |
| | 781 gactttgacc ctgatcctcg gagccggtac accttctgga ctttcatagt gggtggcaca |
| | 841 ctggtgtggc tctccatgta cggtgtgaac caagcccagg tacagcgcta tgtggcctgc |
| | 901 cacacagagg gaaaggccaa actggccctg cttgtcaacc agctgggcct cttcctgatt |
| | 961 gtggccagtg cagcttgctg tggcattgtc atgttcgtct actacaagga ctgtgacccc |
| | 1021 ctcctcacag gccgtatctc agcccccgac cagtacatgc cgctgcttgt gttggacatt |
| | 1081 tttgaggatc tgcccggagt ccccgggctc ttcctggcct gtgcctacag tggcaccctc |
| | 1141 agcactgcat ccaccagcat caacgccatg gcagctgtga ctgtggaaga cctcatcaag |
| | 1201 ccgaggatgc ctggcctggc acctcggaag ttggttttca tctctaaagg gctctcattc |
| | 1261 atctacggct ctgcctgcct cactgtggct gctctgtcct cactgctggg aggtggtgtc |
| | 1321 ctccagggtt ccttcactgt gatgggtgtc atcagtgggc ctctactagg cgccttcacg |
| | 1381 cttgggatgc tgctcccagc ctgcaacacg ccaggcgttc tctccgggtt ggcagcaggc |
| | 1441 ttggctgtat ccctgtgggt ggccgtaggg gccacactgt atccccctgg agagcagacc |
| | 1501 atgggggtgc tgcccacctc ggctgcaggc tgcaccaacg attcggtcct cctgggccca |
| | 1561 cctggagcca ccaacgcttc caacgggatc cccagttctg gaatggacac gggccgccct |
| | 1621 gccctcgctg atacctttta cgccatctcc tatctctatt acggggctct gggcacgctg |
| | 1681 accaccatgc tttgcggtgc tctcatcagc taccttactg gtcccaccaa gcgcagctcc |
| | 1741 ctgggtcccg gattgctgtg gtgggacctt gctcgacaga cagcgtctgt ggccccaaag |
| | 1801 gaagacactg ccaccctgga ggagagcctg gtgaagggac cggaagacat ccctgctgtg |
| | 1861 accaagaagc cccctggcct caagccaggc gccgagaccc accccctgta tctggggcac |
| | 1921 gatgtggaga ccaacctctg agggtggggt ccaagaaggc caatcacagg cctcgggcca |
| | 1981 gcagcctcct ctctggatgg ttggacctga gcatatatag aagcttggct gatacatgcc |
| | 2041 ctgcccagaa gtccctgtgt cttacccgca ccaaagagag agagagagcg agagagagag |
| | 2101 agagagagac agagagagag agagagagag agagacagag agagagagag agagagagag |
| | 2161 agagagagag aggagttggt tctccatcca caaaggaaac cgtctggaac cttcatgccc |
| | 2221 ttgtagattt cagtaggcag cggagaacac tcagcttctc cagactgagg ttttctcatt |
| | 2281 tatcaggcag agaaaccgag ggctgtcacc ccaacaccgg ggaggagaca gtagaagggt |
| | 2341 catagataca aagaaaacta aggcagaggg agaaatgaat tgtctacaga gcacagagct |
| | 2401 ccaaggattg tgaagctacc ttgaggtgcc aagggacgga ttctcagagc cttcacaaga |
| | 2461 cacaaacgga cgagttgcct cctccagttc agatggtttg cagactatca gagaacatgt |
| | 2521 ttctcctgtg atcagctacc tagcctctgc caacgtgttc cagcttccag gaggccacac |
| | 2581 agaccccacc ccccatgctc tcacccttta cccctgtgct tttcacacac taggcaactg |
| | 2641 ctccaccaca ggacctcaca cctagacctc cgtttttgac acagggcctt aaggtagtct |
| | 2701 ggctgccatc tgactatctc tcagcacgtt cacgtgtata atatttcatt ctttttcatt |
| | 2761 gccaagttgt cttgtaagga gagaccacaa tgtgtcatcc atgcccagct tttgtgtcta |
| | 2821 acaaataaaa tcgctgaagg tgttcaaaaa aaaaaaaaaa aa (SEQ ID NO: 5) |
| NP_44 | MEGAEAGARATFGAWDYGVFATMLLVSTGIGLWVGLARGGQRSADDFFTGGRQLAAVPVGLSLAASFMSAVQV |
| 3215. | LGVPAEAARYGLKFLWMCAGQLLNSLLTAFLFLPIFYRLGLTSTYQYLELRFSRAVRLCGTLQYLVATMLYTG |
| 2 | IVIYAPALILNQVTGLDIWASLLSTGIICTLYTTVGGMKAVVWTDVFQVVVMLVGFWVILARGVILLGGPRNM |
| | LSLAQNHSRINLMDFDPDPRSRYTFWTFIVGGTLVWLSMYGVNQAQVQRYVACHTEGKAKLALLVNQLGLFLI |
| | VASAACCGIVMFVYYKDCDPLLTGRISAPDQYMPLLVLDIFEDLPGVPGLFLACAYSGTLSTASTSINAMAAV |
| | TVEDLIKPRMPGLAPRKLVFISKGLSFIYGSACLTVAALSSLLGGGVLQGSFTVMGVISGPLLGAFTLGMLLP |
| | ACNTPGVLSGLAAGLAVSLWVAVGATLYPPGEQTMGVLPTSAAGCTNDSVLLGPPGATNASNGIPSSGMDTGR |
| | PALADTFYAISYLYYGALGTLTTMLCGALISYLTGPTKRSSLGPGLLWWDLARQTASVAPKEDTATLEESLVK |
| | GPEDIPAVTKKPPGLKPGAETHPLYLGHDVETNL (SEQ ID NO: 6) |

In some embodiments, an NIS suitable for use in accordance with the present disclosure has an amino acid sequence of SEQ ID NO:2 (human), SEQ ID NO:4 (mouse), or SEQ ID NO:6 (rat). In some embodiments, a suitable NIS protein may be a homologue or analogue of the human, mouse, or rat NIS protein. For example, homologue or an analogue of a human, mouse or rat protein may be a modified human, mouse or rat protein containing one or more amino acid substitutions, deletions, and/or insertions as compared to a wild-type or naturally-occurring NIS (e.g., SEQ ID NO:2, SEQ ID NO:4, or SEQ ID NO:6), while retaining substantial NIS protein activity. In some embodiments, an NIS protein has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:2 (human), SEQ ID NO:4 (mouse), or SEQ ID NO:6.

In some embodiments, an NIS suitable for use in accordance with the present disclosure is encoded by a nucleic acid sequence of SEQ ID NO:1 (human), SEQ ID NO:3 (mouse), or SEQ ID NO:5 (rat). In some embodiments, an NIS is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:1 (human), SEQ ID NO:3 (mouse), or SEQ ID NO:5. It will be appreciated that nucleic acid sequences of the present disclosure may be derived from a variety of sources including DNA, cDNA, synthetic DNA, synthetic RNA or combinations thereof. Such sequences may comprise genomic DNA which may or may not include naturally occurring introns. Moreover, such genomic DNA may be obtained in association with promoter regions and/or poly (A) sequences. The sequences, genomic DNA or cDNA may be obtained in any of several ways. Genomic DNA can be extracted and purified from suitable cells by means well known in the art. Alternatively, mRNA can be isolated from a cell and used to produce cDNA by reverse transcription or other means.

Nucleic acid constructs for NIS stable transgenic animal generation may include a promoter region, such as an inducible or constitutive promoter. In some embodiments, the NIS constructs do not include a promoter region, and gene expression is instead to be driven by an endogenous promoter. NIS constructs may be designed for random integration, or for targeted integration into the genome of the animal. NIS constructs may include additional features, such as, for example, poly(A) tail, selectable marker(s), introns, among others.

It will be appreciated that expression of NIS may be directed in any animal cell or tissue, thereby permitting detection of that cell or tissue, e.g., using methods and compositions provided herein. Exemplary cells and tissues include, but are not limited to, connective tissue (e.g., blood (e.g., white blood cells, red blood cells, platelets), cartilage, tendons, ligaments, bone, adipose, fibroblasts), muscle tissue (e.g., smooth muscle, skeletal muscle, cardiac muscle), nervous tissue (e.g., central nervous system, peripheral nervous system, brain, spinal cord, cranial nerves, spinal nerves, motor neurons, glial cells), and epithelial tissue (e.g., skin, airway, reproductive tract, digestive tract lining, mesothelia, endothelia).

### Transgenic Animals

Transgenic animals may be generated by any available method, e.g., as discussed in Haruyama et al. Overview: Engineering transgenic constructs and mice. Curr Protoc Cell Biol. March 2009 Chapter:Unit—19:10. For example, a transgenic animal that expresses NIS from a recombinant construct may be produced by introducing the construct into a zygote, an embryonic stem cell, or another multipotent cell derived from the appropriate organism.

“Embryonic stem cells” or “ES cells” as used herein are cells or cell lines usually derived from embryos which are pluripotent meaning that they are undifferentiated cells. These cells are also capable of incorporating exogenous DNA by homologous recombination and subsequently developing into any tissue in the body when incorporated into a host embryo. It is possible to isolate pluripotent cells from sources other than embryonic tissue by methods which are well understood in the art.

Embryonic stem cells in mice have enabled researchers to select for transgenic cells and perform gene targeting. This allows more genetic engineering than is possible with other transgenic techniques. For example, mouse ES cells are relatively easy to grow as colonies in vitro. The cells can be transfected by standard procedures and transgenic cells clonally selected by antibiotic resistance. See, for example, Doetschman et al., 1994, Gene transfer in embryonic stem cells. In Pinkert (Ed.) Transgenic Animal Technology: A Laboratory Handbook. Academic Press Inc., New York, pp. 115-146. Furthermore, the efficiency of this process is such that sufficient transgenic colonies (hundreds to thousands) can be produced to allow a second selection for homologous recombinants. Mouse ES cells can then be combined with a normal host embryo and, because they retain their potency, can develop into all the tissues in the resulting chimeric animal, including the germ cells. The transgenic modification can then be transmitted to subsequent generations.

Methods for deriving embryonic stem (ES) cell lines in vitro from early preimplantation mouse embryos are well known. See for example, Evans et al., 1981 Nature 29:154-156 and Martin, 1981, Proc. Nat. Aca. Sci. USA, 78:7634-7638. ES cells can be passaged in an undifferentiated state, provided that a feeder layer of fibroblast cells or a differentiation inhibiting source is present.

The term “somatic cell” indicates any animal or human cell which is not a sperm or egg cell or is capable of becoming a sperm or egg cell. The term “germ cell” or “germ-line cell” refers to any cell which is either a sperm or egg cell or is capable of developing into a sperm or egg cell and can therefore pass its genetic information to offspring. The term “germ cell-line transgenic animal” refers to a transgenic animal in which the genetic information was incorporated in a germ line cell, thereby conferring the ability to transfer the information to offspring. If such offspring in fact possess some or all of that information, then they are transgenic animals.

The genetic alteration of genetic information may be foreign to the species of animal to which the recipient belongs, or foreign only to the particular individual recipient. In the last case, the altered or introduced gene may be expressed differently than the native gene.

In some embodiments, an embryonic stem cell is harvested and then transformed with a nucleic acid construct encoding a protein of interest, e.g., NIS protein. Transformed embryonic stem cells are injected into inner cell mass of blastocysts, and then the embryo may be transferred into a pseudopregnant animal. In other embodiments, freshly fertilized eggs are harvested before the sperm head has become a pronucleus. The male pronucleus may be injected with the nucleic acid construct encoding a protein of interest, e.g., NIS protein, and the resulting embryo is then implanted into a pseudopregnant animal.

For example, microinjection techniques utilize embryonal cells at various stages of development, according to correspondingly different techniques. Where the zygote is used, micro-injection is a useful technique as described in U.S. Pat. No. 4,873,191, the entire contents of which is herein incorporated by reference. In the mouse, injection of 1-2 picoliters (pl) of DNA solution can be made when the male pronucleus reaches a diameter of approximately 20 micrometers. Furthermore, it is possible to inject the zygote prior to first cleavage, thereby ensuring incorporation of the construct into all somatic and germ cells of the transgenic animal (Brinstei, et al. (1985) Proc. Natl. Acad. Sci. USA 82, 4438-4442). The resulting transgenic mammal will be capable of transmitting the foreign DNA to future offspring. Moreover, in this embodiment it is not necessary to first introduce the targeting construct into a self-replicating plasmid or virus.

In another embodiment, retroviral infection is used to introduce a transgene into a non-human mammal. The technique of retroviral infection uses embryos which have been cultured in vitro to the blastocyst stage, and targets the blastomeres for infection (Jaenich (1976) Proc. Natl. Acad. Sci USA 73:1260-1264). Enzymatic treatment removes the zona pellucida of the blastocysts and facilitates infection via a replication-defective retrovirus carrying the transgene (Van der Putten, et al. (1985) Proc. Natl. Acad. Sci. USA 82, 6148-6152). The transfected blastomeres are then cultured on a monolayer of virus-producing cells. In addition, virus or virus-producing cells can be injected into the blastocoel (Jahner et al. (1982) Nature, 298:623-628). In this embodiment, the resulting transgenic mammals will be mosaic for the transgene, since only a subset of the cells will have incorporated the transgene. In addition, retroviral insertion of the transgene may occur at different positions in the genome which generally will segregate in the offspring. In slight variation of this technique, it is also possible to introduce the transgenes into the germline via intrauterine retroviral infection of the midgestation embryo and thereby generate more comprehensive integration of the transgene (Jahner et al. (1982)).

In another embodiment, the transgene containing the targeting construct is introduced to the ES cell by electroporation (Toneguzzo et al., (1988) Nucleic Acids Res., 16:5515-5532; Quillet et al. (1988) J. Immunol., 141:17-20; Machy et al. (1988) Proc. Natl. Acad. Sci. USA, 85:8027-8031). The cells are then cultured and selected for cells which have successfully integrated the transgene, as described above (e.g., neo in G418 medium). Alternatively, the transgene may be detected by radiolabelled nucleotides, or by other assays of detection which do not require the expression of the selectable marker sequence, such as by PCR amplification techniques.

### Probes and Methods for Detection of NIS in Transgenic Animals

Transgenic animals described herein may be tested to determine NIS transcription and/or translation by any available method, e.g., as discussed below.

### Probes and Methods for Detection of Translation Products

Probe-based methods, include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF) mass spectrometry, microcytometry, microarray, microscopy, fluorescence activated cell sorting (FACS), flow cytometry, laser scanning cytometry, hematology analyzer and assays based on a property of the protein including but not limited to DNA binding, ligand binding, or interaction with other protein partners.

The translation product or polypeptide can be detected and quantified by any of a number of means well known to those of skill in the art. These can include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, immunohistochemistry and the like. A skilled artisan can readily adapt known protein/antibody detection methods for use in determining the expression level of one or more biomarkers in a serum sample.

### Probes and Methods for Detection of Transcription Products

Translational expression can be assessed by any of a wide variety of well known methods for detecting expression. Non-limiting examples of such methods include nucleic acid hybridization methods, nucleic acid reverse transcription methods, and nucleic acid amplification methods.

In certain embodiments, activity of a particular gene is characterized by a measure of gene transcript (e.g., mRNA). Detection can involve quantification of the level of gene expression (e.g., cDNA, mRNA), or, alternatively, can be a qualitative assessment of the level of gene expression, in particular in comparison with a control level. The type of level being detected will be clear from the context.

Methods of detecting and/or quantifying the gene transcript (mRNA or cDNA made therefrom) using nucleic acid hybridization techniques are known to those of skill in the art (see e.g., Sambrook et al. supra). For example, one method for evaluating the presence, absence, or quantity of cDNA involves a Southern transfer as described above. Briefly, the mRNA is isolated (e.g., using an acid guanidinium-phenol-chloroform extraction method, Sambrook et al.) and reverse transcribed to produce cDNA. The cDNA is then optionally digested and run on a gel in buffer and transferred to membranes. Hybridization is then carried out using the nucleic acid probes specific for the target cDNA.

The isolated nucleic acid can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

An alternative method for determining the level of a transcript involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. Fluorogenic rtPCR can also be used in the methods of the invention. In fluorogenic rtPCR, quantitation is based on amount of fluorescence signals, e.g., TaqMan and sybr green. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations can be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a healthy subject, or between samples from different sources.

### Detectable Substrates or Moieties

As discussed above, the NIS protein is a transmembrane glycoprotein which allows cells to take up anions, such as iodide, e.g., for use in biosynthetic processes. NIS activity also permits tissues expressing the protein to take up various detectable moieties, e.g., radioactive, anion isotopes, e.g., iodine (¹²³I, ¹²⁴I, ¹²⁵I, ¹³¹I, pertechnetate [^{99m}Tc] and perrhenate. [¹⁸⁸Re]. For example, NIS-expressing cells or tissue can be imaged when an animal is given a free radioactive anion form of radioactive iodine and/or technetium. It will be appreciated that detectable moieties, such as radioactive iodine and technetium anions, may be used in non-invasive imaging as covalent or chelated complexes with other molecules.

Radioactive isotopes of iodine include, but are not limited to, ¹²³I, ¹²⁴I, ¹²⁵I, and ¹³¹I. For example, ¹²³I has a half-life of about 13.22 hours, and the decay by electron capture to ¹²³tellurium emits gamma radiation with a predominant energy of 159 keV, which can be detected by gamma cameras. ¹³¹I, also called radioiodine, has a half-life of about 8 days, and gives off about 10% of its energy via gamma radiation, which can be detected by various nuclear medicine imaging techniques.

⁹⁹mTc is a generator-produced nuclide with a T_{1/2}=6 hours. ^{99m}Tc yields relatively high energy photons which can easily penetrate body tissues (140 keV g-rays). ^{99m}Tc is characterized by a relatively low radiation dose per mCi, so a patient can generally receive a maximum of 40-50 mCi, e.g., for a stress/rest study. ^{99m}Tc-Sestamibi, ^{99m}Tc-Tetrofosmin, and ^{99m}Tc-Teboroxime have been approved as commercial radiopharmaceuticals, e.g., for myocardial perfusion imaging in nuclear cardiology (Kim, et al. World J Hepatol 2(1):21-31, 2010). These cationic ^{99m}Tc radiotracers are highly lipophilic with cationic or neutral charge, contain at least two ether-like linkages (N—O—R or C—O—R), and are excreted though the hepatobiliary system due to their high lipophilicity (Kim, et al. World J Hepatol 2(1):21-31, 2010).

The pertechnetate (technetate(VII)) ion, which is an oxoanion with a chemical formula TcO₄⁻, may be used as a water soluble carrier of an isotope, e.g., the ^{99m}Tc isotope. Pertechnetate is widely used in nuclear medicine, as it can substitute for iodine in the NA/I Symporter (NIS) Channel, e.g., in follicular cells of the thyroid gland. Indeed, pertechnetate acts to inhibit uptake of iodine into the follicular cells. Pertechnetate is also actively accumulated and secreted by the mucoid cells of the gastric mucosa. Exemplary uses of pertechnetate in nuclear medicine include, but are not limited to, thyroid imaging; evaluation of testicular torsion; labeling of autologous red blood cells, e.g., for MUGA (Multi Gated Acquisition Scan) scans to evaluate left ventricular cardiac function; localization of gastrointestinal bleeding, e.g., prior to embolization or surgical management; in damaged red blood cells, e.g., to detect ectopic splenic tissue; and in looking for ectopic gastric tissue, e.g., as found in a Meckel's diverticulum with Meckel's Scans.

Rhenium-188 (¹⁸⁸Re) is a generator-derived radionuclide which emits beta and gamma particles, e.g., as disclosed in Pillai et al. Curr Radiopharm 5(3):228-43 (2012). ¹⁸⁸Re has a half-life of about 17 hours, and has a β-ray emission of sufficient energy (2.11 MeV) to penetrate 5-10 mm of thickened synovial membrane, and a low-level γ-ray emission (155 keV) which makes scintigraphic monitoring possible, without harming patients or practitioners.

Medical imaging techniques that rely on detection of emissions from tracers originating from within the body of the subject being imaged are widely used for diagnosis of various diseases. Nuclear physics-based molecular imaging techniques, such as positron emission tomography (PET) and single photon emission computed tomography (SPECT) allow functional imaging of subjects at the molecular level based on the use of radioactive isotopes. For example, SPECT is based on the use of radioisotopes that emit gamma rays and PET is based on the use of radioisotopes that emit positrons, which annihilate to produce gamma rays.

One of ordinary skill in the art will recognize that different radiopharmaceuticals display different pharmacokinetic properties, such as elimination, clearance from and/or accumulation in biological tissues, and half-life (T_{1/2}). Commercially available radiopharmaceuticals are widely used and may be appended onto biologically relevant molecules by chemical synthesis techniques well known in the art. Typically, the half-lives of radiotracers used in imaging are relatively short, and thus many cyclotrons are key features of radiotracer detection apparatuses, such as PET and SPECT scanners, or gamma cameras. Exemplary radionuclides that can be used as detectable moieties, include, but are not limited to, technetium (e.g., ^{99m}Tc), indium (e.g., ¹¹¹In), cobalt (e.g., ⁵⁷Co), chromium (e.g., ⁵¹Cr), thallium (e.g., ²⁰¹Tl), gallium (e.g., ⁶⁷Ga), iodine (e.g., ¹²³I, ¹²⁴I, ¹²⁵I, ¹³¹I), rhenium (e.g., ¹⁸⁸Re), lutetium (e.g., ¹⁷⁷Lu) and samarium (e.g., ¹⁵³Sm).

Nuclear probes that may be used in accordance with the present invention may comprise single-photon gamma emitters or positron emitters. There are many single-photon gamma emitters in the range of 50 to 300 keV. Numerous FDA approved radiopharmaceuticals employ these radioisotopes.

It will be appreciated that provided radiopharmaceuticals may be administered singularly, or in combination with one or more radiopharmaceuticals.

### Detection

Several techniques can be used to detect the agents described herein. Examples of such techniques include single photon emission computed tomography (SPECT), Positron Emission Tomography (PET), Magnetic Resonance Imaging (MRI) and scintigraphy.

### SPECT

Isotopes that decay by electron capture and/or γ emissions can be directly detected by SPECT. Certain proton-rich radionuclides, such as ¹²³I and ^{99m}Tc, may instead capture an orbiting electron, once again transforming a proton to a neutron (Sorenson J A, and Phelps M E. Philadelphia: W. B. Saunders; 1987). The resulting daughter nucleus often remains residually excited. This meta-stable arrangement subsequently dissipates, thereby achieving a ground state and producing a single γ photon in the process. Because γ photons are emitted directly from the site of decay, no comparable theoretical limit on spatial resolution exists for SPECT. However, instead of coincidence detection, SPECT utilizes a technique known as collimation (Jaszczak R J. Boca Raton: CRC Press; (1991): 93-118). A collimator may be thought of as a lead block containing many tiny holes that is interposed between the subject and the radiation detector. Given knowledge of the orientation of a collimator's holes, the original path of a detected photon is linearly extrapolated and the image is reconstructed by computer-assisted tomography.

Simultaneous dual-radionuclide studies can be performed with SPECT. SPECT permits identification of isotopes based on the energies of emitted photons, which allows for simultaneous dual-isotope imaging where distributions of the two isotopes are differentiated by setting multiple energy windows during image acquisitions (Shcherbinin, et al. Phys. Med. Biol. 57:4755-4769, 2012). However, the separation of tracers in positron emission tomography

(PET) is more difficult because the annihilation photons created by the PET radiotracers always have the same energy (Rahmim, et al., Nucl. Med. Commun. 29:193-207, 2008).

Quantitative dual-isotope SPECT imaging is a useful tool that can be used to quantify and compare the biodistribution of different ligands, each labeled with a different radionuclide, in the same animal. Since the biodistribution results are not blurred by experimental or physiological inter-animal variations, this approach allows determination of each of the ligand's net targeting effect. However, dual-isotope quantification may potentially be complicated by crosstalk between the two radionuclides used. (Hinjen, et al. Contrast Media Mol Imaging 7(2):214-22, 2012) Various quantitative dual-isotope SPECT protocols have been developed to combine different radionuclides in the same animal, including, but not limited to: ^{99m}Tc and ²⁰¹Tl (de Jong, et al., Eur. J. Nucl. Med. Mol. Imag. 29:1063-71, 2002; Kadrmas, et al., Phys. Med. Biol. 44:1843-60, 1999; Song, et al., IEEE Trans. Nucl. Sci. 51:72-9, 2004); ¹¹¹In and ¹⁷⁷Lu (Hijnen, et al., Contrast Media Mol Imaging, 7(2):214-22, 2012); ¹¹¹In and a ^{99m}Tc (James Brice, Dual-isotope SPECT/CT finds infections in diabetic feet. Diagnostic Imaging, Jan. 1, 2009; Du, et al., J. Nucl. Med. 49 (Suppl. 1):152, 2008); ¹²³I and ^{99m}Tc (Shcherbinin, et al. Phys. Med. Biol. 57:4755-4769, 2012; Ouyang, et al. Med. Phys. 34:3263-72, 2007; Ouyang, et al., Med. Phys. 36:602-11, 2009; Du, et al., Med. Phys. 34:3530-43, 2007; Du, et al., Med. Phys. 36:2021-33, 2009).

### Positron Emission Tomography (PET)

Positron Emission Tomography (PET) is a nuclear medicine imaging technique that detects pairs of gamma rays emitted indirectly by a positron-emitting radionuclide to form a three-dimensional image. In some cases, three dimensional imaging and analysis is aided by a CT X-ray scan that may be performed on the patient during the same session in the same machine. The emitted positrons from the decaying radioisotope travel in tissue of the subject for a short distance, during which time the positrons lose kinetic energy. The positrons decelerate to a point where they are able to interact with an electron, which annihilates both electron and positron, producing a pair of annihilation (gamma) photons moving in opposite directions. The photons are detected by a scintillator in the PET device. Statistical analysis and image reconstruction are performed in order to generate a three dimensional image of the tissue.

### Magnetic Resonance Imaging (MRI)

Magnetic Resonance Imaging (MRI) is a medical technique using nuclear magnetic resonance to image nuclei of atoms inside the body. An MRI scanner has a large, powerful magnet where the magnetic field is used to align the magnetization of atomic nuclei in the body, and radio frequency magnetic fields are applied to systematically alter the alignment of the magnetization, causing the nuclei to produce a rotating magnetic field detectable by the scanner. Magnetic field gradients in different directions permit two dimensional images or three dimensional volumes to be obtained. Unlike other imaging techniques, such as CT scans or X-rays, MRI does not use ionizing radiation.

### Scintigraphy

Scintigraphy is a diagnostic test used in nuclear medicine in which a radiopharmaceutical agent is administered to a subject, and the emitted radiation of captured by external detectors, e.g., gamma cameras, to form two-dimensional images. Scinitgraphy analyses can be performed on various tissues and regions of the body, including, but not limited to, biliary system, lungs, bone, heart, parathyroid, thryroid, and full body. Scintigraphy differs from SPECT and PET, which each form 3-dimensional images.

One advantage of the transgenic animals of the present invention is that they may be imaged while they are living. It will be appreciated that animals may be imaged at a single time interval, or at multiple time intervals during their lives, e.g., at 1, 2, 3, 4, 5, 6, 7, 8, 9, 10 or more time intervals.

### Developmental Models, Disease Models, and Screening

Among other things, transgenic animals expressing NIS can be used as models for studying development and/or physiological responses in the animal. It will be appreciated that any developmental pathway or physiological response may be studied in provided transgenic animals, including, but not limited to, neurological development, hypoxia response, oxidative stress response, myelination and demyelination responses, astrocyte response (e.g., glial scar formation), among others. Various assays, such as functional assays, behavioral assays, physiological assays, e.g., hormonal assays, electrophysiological assays, ion transport assays, neuronal function assays, may be used to study the development and/or physiological responses in the animal.

Transgenic animals provided herein may be used to screen or test the efficacy, safety and/or toxicity of various test compounds for treatment of a disease, disorder or condition. Such animal models can provide insight into the mechanism of action of the compound, as well as provide information for clinical trials of the compound in humans. A test compound, e.g., pharmaceutical compound, may be administered to the transgenic animal by any available route. For example, compositions can be administered by an enteral, topical, or parenteral mode (e.g., intravenous, subcutaneous, intraperitoneal, or intramuscular injection). The phrases “parenteral administration” as used herein refers to a mode of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Compounds may be administered at any appropriate dose. Compounds may be administered in a single dose, or in multiple doses at a predetermined time interval. A test compound may be a protein, peptide, antibody, or small molecule, among other things.

Provided transgenic animals may also be used to study development, progression and/or treatment of a disease, disorder or condition. It will be appreciated that NIS transgenic disease model animals may be generated by any available method. For example, an NIS transgenic animal may be administered a compound, e.g., a pharmaceutical compound, or a treatment, e.g., exposure to a disease inducing agent, to induce disease. In another example, an NIS transgenic animal may be crossed with any available disease model animals, to generate progeny which ectopically express NIS and serve as a disease model. Exemplary animal disease models, e.g., animals capable of being induced to develop or having a genetic predisposition to developing a certain disease, disorder, or condition, include animal models of cancer, heart disease, hypertension, metabolic and hormonal disorders, diabetes, obesity, osteoporosis, glaucoma, skin pigmentation diseases, blindness, deafness, neurodegenerative disorders (e.g., CNS demyelinating diseases, CNS injury, Amyotrophic lateral sclerosis (ALS), Huntington's disease, Alzheimer's disease, Parkinson's disease, diabetic neuropathy, stroke, idiopathic inflammatory demyelinating disease, multiple sclerosis (MS), optic neuritis (e.g., acute optic neuritis), neuromyelitis optica (NMO), leukodystrophies, vitamin B12 deficiency, progressive multifocal leukoencephalopathy (PML), encephalomyelitis (EPL), acute disseminated encephalomyelitis (ADEM), central pontine myelolysis (CPM), Wallerian Degeneration, adrenoleukodystrophy, Alexander's disease, Pelizaeus Merzbacher disease (PMZ), traumatic glaucoma, periventricular leukomalatia (PVL), or transverse myelitis), psychiatric disturbances (e.g., anxiety or depression), and birth defects (e.g., cleft palate or anencephaly). Various animal models for CNS disorders are disclosed, e.g., in McGonigle P Biochem Pharmacol Animal Models of CNS Disorders (2013). Exemplary diseases, including Amyotrophic Lateral Sclerosis (ALS) and Multiple Sclerosis (MS) are discussed in further detail below.

### Amyotrophic Lateral Sclerosis (ALS)

Amyotrophic Lateral Sclerosis (ALS), also known as Lou Gehrig's disease, is characterized by rapidly progressing weakness, muscle atrophy and fasciculations, muscle spasticity, difficulty speaking (dysarthria), difficulty swallowing (dysphagia), and difficulty breathing (dyspnea). The order and rate of symptoms of ALS varies from person to person, but ultimately most patients lose the ability to walk or use their hands and arms, and breathing and eating problems lead to pneumonia and weight loss. The rate of ALS progression can be measured using a standard outcome measure “ALS Functional Rating Scale (Revised).” In approximately 95% of ALS cases, no family history of the disease is present and there is no known cause for the disease. In familial ALS, genetic abnormalities have been identified, such as mutations in the superoxide dismutase (SOD1) gene. Mutations in several genes have been linked to various types of ALS. Pathophysiologically, ALS is characterized by death of both upper and lower motor neurons in the motor cortex of the brain, the brain stem, and the spinal cord. Prior to the destruction of motor neurons, they develop proteinaceous inclusions in their cell bodies and axons.

ALS diagnosis is usually not definitive, and generally requires a neurologic examination at regular intervals to confirm a diagnosis. Tests that are generally performed in order to exclude the possibility of a condition other than ALS include electromyography (EMG), nerve conduction velocity (NCV), magnetic resonance imaging (MRI), blood tests, and urine tests, among others.

Current therapies for ALS are limited, and most current therapies are designed to relieve symptoms and improve quality of life for patients. For example, Riluzole (Rilutek) is a treatment that has been found to improve survival, however, only to a modest extent. Several drugs are currently in clinical trials, including thalidomide, lenalidomide, and dexpramipexole (KNS-760704). Exemplary symptom management treatments for ALS include, but are not limited to, medications to reduce fatigue, ease muscle cramps, control spasticity (e.g., spasmolytic (anti-spastic) agents, such as baclofen, diazepam, tizanidine, and dantrolene), reduce excess saliva and phlegm, alleviate pain, depression, sleep disturbances, dysphagia (e.g., trihexyphenidyl, amitriptyline), and constipation.

### Multiple Sclerosis

Multiple sclerosis (MS) is a central nervous system disease that is characterized by inflammation and loss of myelin sheaths. Patients having MS can be identified by clinical criteria establishing a diagnosis of clinically definite MS as defined by Poser et al., Ann. Neurol. 13:227, 1983. Briefly, an individual with clinically definite MS has had two attacks and clinical evidence of either two lesions or clinical evidence of one lesion and paraclinical evidence of another, separate lesion. Definite MS may also be diagnosed by evidence of two attacks and oligoclonal bands of IgG in cerebrospinal fluid or by combination of an attack, clinical evidence of two lesions and oligoclonal band of IgG in cerebrospinal fluid. The McDonald criteria can also be used to diagnose MS. (McDonald et al., 2001, Recommended diagnostic criteria for Multiple sclerosis: guidelines from the International Panel on the Diagnosis of Multiple Sclerosis, Ann Neurol 50:121-127). The McDonald criteria include the use of MRI evidence of CNS impairment over time to be used in diagnosis of MS, in the absence of multiple clinical attacks. Effective treatment of multiple sclerosis may be evaluated in several different ways. The following parameters can be used to gauge effectiveness of treatment. Two exemplary criteria include: EDSS (extended disability status scale), and appearance of exacerbations on MRI (magnetic resonance imaging).

The EDSS is a means to grade clinical impairment due to MS (Kurtzke, Neurology 33:1444, 1983). Eight functional systems are evaluated for the type and severity of neurologic impairment. Briefly, prior to treatment, patients are evaluated for impairment in the following systems: pyramidal, cerebella, brainstem, sensory, bowel and bladder, visual, cerebral, and other. Follow-ups are conducted at defined intervals. The scale ranges from 0 (normal) to 10 (death due to MS). A decrease of one full step indicates an effective treatment (Kurtzke, Ann. Neurol. 36:573-79, 1994), while an increase of one full step will indicate the progression or worsening of disease (e.g., exacerbation). Typically patients having an EDSS score of about 6 will have moderate disability (e.g., walk with a cane), whereas patients having an EDSS score of about 7 or 8 will have severe disability (e.g., will require a wheelchair).

Exacerbations are defined as the appearance of a new symptom that is attributable to MS and accompanied by an appropriate new neurologic abnormality (IFNB MS Study Group, supra). In addition, the exacerbation must last at least 24 hours and be preceded by stability or improvement for at least 30 days. Briefly, patients are given a standard neurological examination by clinicians. Exacerbations are mild, moderate, or severe according to changes in a Neurological Rating Scale (Sipe et al., Neurology 34:1368, 1984). An annual exacerbation rate and proportion of exacerbation-free patients are determined.

Exemplary symptoms associated with multiple sclerosis include: optic neuritis, diplopia, nystagmus, ocular dysmetria, internuclear opthalmoplegia, movement and sound phosphenes, afferent pupillary defect, paresis, monoparesis, paraparesis, hemiparesis, quadraparesis, plegia, paraplegia, hemiplegia, tetraplegia, quadraplegia, spasticity, dysarthria, muscle atrophy, spasms, cramps, hypotonia, clonus, myoclonus, myokymia, restless leg syndrome, footdrop, dysfunctional reflexes, paraesthesia, anaesthesia, neuralgia, neuropathic and neurogenic pain, l'hermitte's, proprioceptive dysfunction, trigeminal neuralgia, ataxia, intention tremor, dysmetria, vestibular ataxia, vertigo, speech ataxia, dystonia, dysdiadochokinesia, frequent micturation, bladder spasticity, flaccid bladder, detrusor-sphincter dyssynergia, erectile dysfunction, anorgasmy, frigidity, constipation, fecal urgency, fecal incontinence, depression, cognitive dysfunction, dementia, mood swings, emotional lability, euphoria, bipolar syndrome, anxiety, aphasia, dysphasia, fatigue, Uhthoff's symptom, gastroesophageal reflux, and sleeping disorders.

Each case of MS displays one of several patterns of presentation and subsequent course. Most commonly, MS first manifests itself as a series of attacks followed by complete or partial remissions as symptoms mysteriously lessen, only to return later after a period of stability. This is called relapsing-remitting MS (RRMS). Primary-progressive MS (PPMS) is characterized by a gradual clinical decline with no distinct remissions, although there may be temporary plateaus or minor relief from symptoms. Secondary-progressive MS (SPMS) begins with a relapsing-remitting course followed by a later primary-progressive course. Rarely, patients may have a progressive-relapsing (PRMS) course in which the disease takes a progressive path punctuated by acute attacks. PPMS, SPMS, and PRMS are sometimes lumped together and called chronic progressive MS.

This invention is further illustrated by the following examples which should not be construed as limiting. The contents of all references, figures, sequence listing, patents and published patent applications cited throughout this application are hereby incorporated by reference.

### EXAMPLES

### Example 1

### Generation of a Transgenic NIS Reporter Animal

This example describes the development of a transgenic sodium iodide symporter (NIS) reporter animal. The use of NIS expression instead of other approaches that use fluorescent, luminescent, or MRI imaging offers a solution for overcoming the current impediments in live animal transgene expression measurements. To date, no transgenic animals have been generated that constitutively express NIS. As described in this example, by directing the expression of NIS alone in specific cells of transgenic animals, one can readily take advantage of the robust imaging capabilities and wide availability of NIS transported isotopes to study various biological processes.

For example, using NIS as a thy-1 driven transgene will allow for highly sensitive high resolution imaging of the NIS expressing neuroanatomical pathways via radioactive iodine or technetium. Moreover, the NIS-expressing transgenic animals can be imaged repeatedly using radioactive iodine and technetium without any surgery or terminal procedures being required. This opportunity to repeatedly and conveniently observe transgene expression is of high value in understanding many biological functions such as the development, maturation and degeneration of organs such as the brain. When coupled with genetic disease backgrounds, the NIS transgenic expression approach will provide important insights into dynamics of pathophysiology and the impact of therapeutic effects.

By linking NIS expression to a specific inducible promoter, regulation of the gene expression mediated by the gene promoter of interest is able to be studied. When expressed under inducible promoters, the NIS transgene will allow for the convenient and highly localized monitoring of several physiological responses. For example, when expressed under the control of promoters containing hypoxia responsive elements (HRE) or antioxidant response elements (ARE), NIS expression patterns will permit monitoring of tissue specific responses to hypoxia and oxidative stress, or drugs that impact hypoxic or antioxidant signaling pathways. Likewise, expression under the proteolipid protein promoter (PLP) will allow for monitoring of myelination and re-myelination responses. Expression under the GFAP promoter allow for monitoring of astrocyte behavior such as glial scar formation.

By linking the expression of NIS to the expression of other genes, the highly sensitive, high resolution imaging properties of SPECT and PET can be used to study the expression of any gene of interest. Indeed, use of the NIS transgene allows innovations made available through reporter transgene technologies to be readily accessible via in vivo high resolution imaging using the high sensitivity of radioactive iodine and technetium.

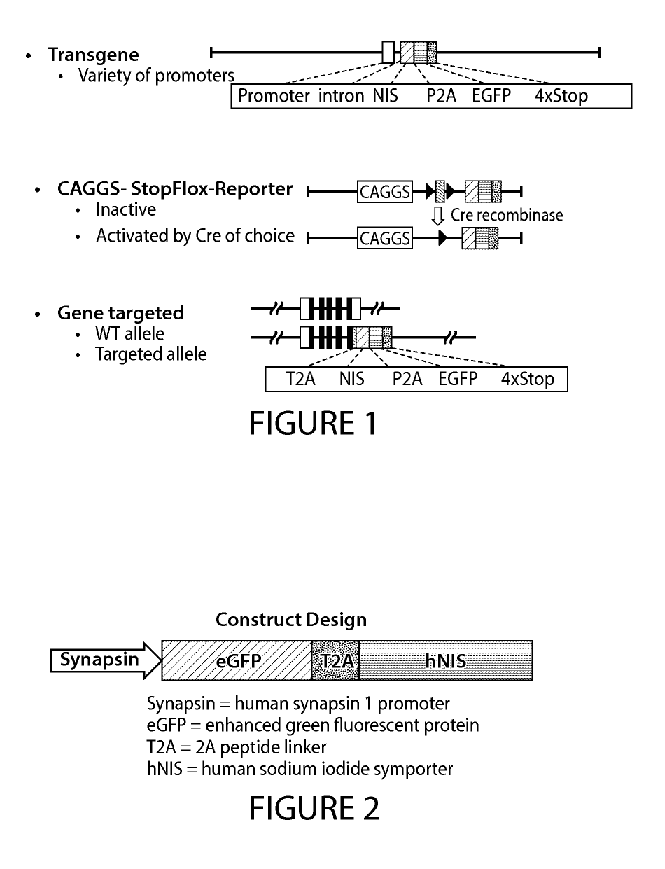

The NIS reporter gene may be delivered for expression in a variety of cell types, in mouse rat or other species in which such a reporter would be useful, and by a variety of stable or transient transgenic delivery mechanisms including viral vectors, and the generation of any transgenes stably integrated into the genome, e.g., as described in FIG. 1. FIG. 1 illustrates three stable transgenic approaches: a) random integration into the genome, for instance by pronuclear micro-injection of transgene DNA into the male pronucleus of a fertilized eggs using standard techniques for generation of transgenic animals; b) integration of an inducible transgene into either a random or a predetermined genomic locus; c) gene targeted integration of the NIS reporter to a gene of interest for expression from the endogenous promoter expressed as a single transcript with, or in place of, the endogenous coding region.

Using a standard transgene construct as in FIG. 1 the transgene construct is comprised of a promoter of interest, a non-coding 5′UTR, an intron (the rabbit betaglobin intron for example) an exon including the entire NIS coding region and a stop codon and 4×-transcritional stop sequence. The transgene may also include any direct reporter, for example green fluorescent protein, GFP, in which the NIS and GFP are co-expressed from the same transcript with an autocatalytic peptide cleavage element (P2A or T2A) to ensure co-translation followed by peptide cleavage separating the NIS and GFP proteins. This approach allows for generation of animal models in which a specific cell type or types expressing the NIS reporter. The secondary reporter provides for rapid confirmatory characterization of cell type expression via ex vivo microscopy.

In the case of integrating an inducible transgene the use of an inducible promoter (as in FIG. 1) a universal promoter, active in most cells in vivo, is used followed by a translational stop and 4×STOP element flanked by recombinase recognition sites (for example LoxP) followed by the dual NIS-GFP transcript as described above for the NCIGFP transgene. The so-called “Stop-FLox” element provides for development of animal models in which the NIS transgene (with or without a secondary reporter) is not transcribed until induction by recombinase-mediated deletion of the STOP-FLox element. This STOP-FLox format provides for tissue-specific and/or temporal control of transgene expression based on the mechanism for delivering the recombinase, for example crossing STOP-FLox-NIS animals with animals transgenic for a tissue specific Cre recombinase.

An alternate approach of targeting the NIS coding region to a gene of interest would include, for example the use of an autocatalytic peptide (like P2A or T2A) to link the NIS reporter gene (with or without a secondary reporter like GFP) as described above to the 3′ end of the coding region (5′ to the endogenous stop codon) of the endogenous target gene. The inducible approach (for instance STOP-FLox) can be added to either the transgenic approach or gene targeted approach. This approach provides for expression and NIS based imaging of cells in a gene specific manner as opposed to a cell type specific manner.

### Example 2

### Generation of NIS Transgenic Mouse Line

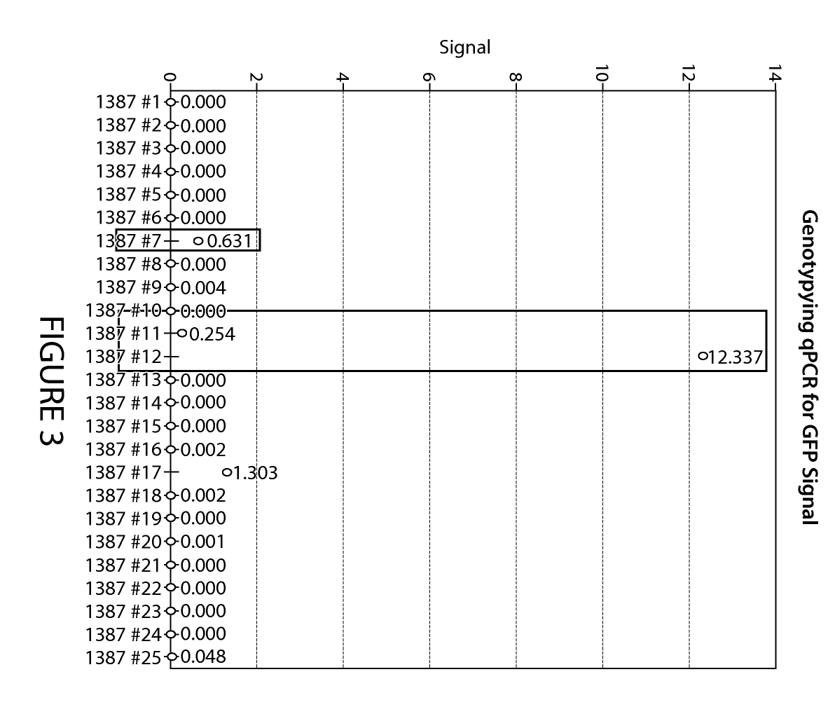

NIS transgenic mice were generated by pronuclear injection of a construct shown in FIG. 2 into fertilized mouse eggs. The transgene construct includes a human synapsin 1 promoter and the genes encoding an enhanced green fluorescent protein (eGFP), a 2A peptide linker (T2A) and a human sodium iodide symporter (hNIS).

F₀ pups were generated and genotyped by quantitative PCR (qPCR) reactions using a GFP probe. The genotyping results are shown in FIG. 3, Three male pups (#7, #11 and #12) were genotyped positive for the transgene and survived to adulthood. Animals were set up with females to breed. Each of the three F₀ pups has generated a litter of F1 pups for further genotyping.

### REFERENCES

1. Kim K., Chung J K, Kang J H, et al. Visualization of Endogenous p53-Mediated Transcription In vivo Using Sodium Iodide Symporter. Clin Cancer Res 2005; 11:123-128.
2. Niva G, Andersone R D, Madsenc M T, Graham M M, Oberley L W, Domanna F W. Dual-expressing adenoviral vectors encoding the sodium iodide symporter for use in noninvasive radiological imaging of therapeutic gene transfer. Nuclear Medicine and Biology 33 (2006) 391-398
3. Jiantu Che, Mikhail Doubrovin, Inna Serganova, Ludmila Ageyeva, Pat Zanzonico, and Ronald Blasberg. hNIS-IRES-eGFP Dual Reporter Gene Imaging Molecular Imaging. Vol. 4, No. 2, April 2005, pp. 128-136
4. Kim K I, Park J J, Lee Y L, Lee T S, Woo K S, Song I, Kim K M, Choi C W, Lim S M & Kang J H. Gamma camera and optical imaging with a fusion reporter gene using human sodium/iodide symporter and monomeric red fluorescent protein in mouse model. International Journal of Radiation Biology, December 2011; 87(12): 1182-1188
5. Kim H K, Jeon Y H, Kang J H, Lee Y J et al., In Vivo Long-Term Imaging and Radioiodine Therapy by Sodium-Iodide Symporter Gene Expression Using a Lentiviral System Containing Ubiquitin C Promoter. Cancer Biology & Therapy 6:7, 1130-1135; July 2007
6. Zhou R, Acton P D, Ferrari V A. Imaging Stem Cells Implanted in Infarcted Myocardium. (J Am Coll Cardiol 2006; 48:2094-106
7. Cho J Y, Shen D H, Yang W, Williams B, Buckwalter T L, La Perle K M, Hinkle G, Pozderac R, Kloos R, Nagaraja H N, Barth R F, Jhiang S M (2002). In vivo imaging and radioiodine therapy following sodium iodide symporter gene transfer in animal model of intracerebral gliomas. Gene Ther. 9:1139-1145.
8. Groot-Wassink T, Aboagye E O, Wang Y, Lemoine N R, Reader A J, Vassaux G (2004). Quantitative imaging of Na/I symporter transgene expression using positron emission tomography in the living animal. Mol Ther. 9:436-442.
9. Groot-Wassink T, Aboagye E O, Glaser M, Lemoine N R, Vassaux G (2002). Adenovirus biodistribution and noninvasive imaging of gene expression in vivo by positron emission tomography using human sodium/iodide symporter as reporter gene. Hum Gene Ther. 13:1723-1735.
10. Smanik P A, Liu Q, Furminger T L, Ryu K, Xing S, Mazzaferri E L, Jhiang S M (1996). Cloning of the human sodium iodide symporter. Biochem Biophys Res Commun. 226:339-345.
11. Dai G, Levy O, Carrasco N (1996). Cloning and characterization of the thyroid iodide transporter. Nature. 379:458-460.
12. Dadachova E, Carrasco N (2004). Na/I symporter (NIS): Imaging and therapeutic applications. Semin Nucl Med. 34:23-31. Review.
13. Barton K N, Tyson D, Stricker H, Lew Y S, Heisey G, Koul S, de la Zerda A, Yin F F, Yan H, Nagaraja T N, Randall K A, Jin G K, Fenstermacher J D, Jhiang S, Ho Kim J, Freytag S O, Brown S L (2003). GENIS: Gene expression of sodium iodide symporter for noninvasive imaging of gene therapy vectors and quantification of gene expression in vivo. Mol Ther. 8:508-158.
14. Tjuvajev J G, Stockhammer G, Desai R, Uehara H, Watanabe H, Gansbacher B, Blasberg R G (1995). Imaging the expression of transfected genes in vivo. Cancer Res. 55:6126-6132.
15. Tjuvajev J G, Joshi A, Callegari J, Lindsley L, Joshi R, Balatoni J, Finn R, Larson S M, Sadelain M, Blasberg R G (1999). A general approach to the non-invasive imaging of transgenes using cis-linked herpes simplex virus thymidine kinase. Neoplasia. 4: 315-320.
16. Hadjantonakis A K, Dickinson M, Fraser S E, Papaioannou V E (2003). Technicolour transgenics: Imaging tools for functional genomics in the mouse. Nat Rev Genet. 4:613-625.
17. Doubrovin M, Ponomarev V, Beresten T, Balatoni J, Bornmann W, Finn R, Humm J, Larson S, Sadelain M, Blasberg R, Gelovani Tjuvajev J G (2001). Imaging transcriptional regulation of p53-dependent genes with positron emission tomography in vivo. Proc Natl Acad Sci USA. 98:9300-9305.
18. Shen D H, Kloos R T, Mazzaferri E L, Jhian S M. Sodium iodide symporter in health and disease. Thyroid 2001; 11:415-25.
19. Geraldes CFGC, Laurent S, Classification and basic properties of contrast agents for magnetic resonance imaging, Contrast Media & Mol Imaging, 4:1-23, 2009.
20. Dohan O, De La Viega A, Paroder V, Riedel C, Artani M, Reed M, Ginter C, Carrasco N, The Sodium/Iodide Symporter (NIS): Characterization, Regulation and Medical Significance, Endocrine Reviews 24(1):48-77, 2003.
21. Klutz K, Willhauck M J, Wunderlich N, Zach C, Anton M, Senekowitsch-Schmidtke R, Goeke B, Spitzweg C, Sodium Iodide Symporter (NIS)-Mediated Radionuclide (131I, 188Re) Therapy of Liver Cancer After Transcriptionally Targeted Intratumoral in vivo NIS Gene Delivery, Human Gene Therapy, 22:1403-1412, November 2011
22. Meikle S R, Kench P, Kassiou M, Banati R B. Small animal SPECT and its place in the matrix of molecular imaging technologies. Phys Med Biol. 50:R45-R61, 2005.

### EQUIVALENTS

Those skilled in the art will recognize, or be able to ascertain using no more than routine experimentation, many equivalents to the specific embodiments of the invention described herein. Such equivalents are intended to be encompassed.

What is claimed is:

1. A transgenic animal having somatic and germ cells comprising a stably integrated nucleic acid for ectopic protein expression in transgenic animal cells of a sequence encoding a sodium iodide symporter (NIS) protein or biologically active fragment thereof.

2. The transgenic animal of claim 1, wherein the animal is selected from a mouse, rat or rabbit.

3. The transgenic animal of claim 1 or 2, wherein the nucleic acid is stably integrated by targeted integration.

4. The transgenic animal of claim 1 or 2, wherein the nucleic acid is stably integrated by random integration.

5. The transgenic animal of any of claims 1-4, wherein the stably integrated nucleic acid comprises a promoter region that is operably linked to the sequence encoding the NIS protein or biologically active fragment thereof.

6. The transgenic animal of claim 5, wherein the promoter region is selected from a human promoter region, a mouse promoter region, a rat promoter region, or a viral promoter region.

7. The transgenic animal of claim 5 or 6, wherein the promoter is a constitutive promoter, e.g., thy-1.

8. The transgenic animal of any of claims 5-7, wherein the promoter comprises an inducible promoter element, e.g., hypoxia responsive element (HRE), antioxidant response element (ARE), proteolipid protein (PLP) promoter, glial fibrillary acidic protein (GFAP) promoter, Stop-FLox element.

9. The transgenic animal of any of claims 1-3, wherein the stably integrated nucleic acid is integrated into the animal cells at a genomic locus such that an endogenous promoter is operably linked to the sequence encoding the NIS protein or biologically active fragment thereof.

10. The transgenic animal of claim 9, wherein the stably integrated nucleic acid is integrated at a genomic locus that is 3′ to the endogenous promoter.

11. The transgenic animal of any of the preceding claims, wherein the NIS protein or biologically active fragment thereof is selected from a human NIS protein, e.g., SEQ ID NO:2, or biologically active fragment thereof, a mouse NIS protein, e.g., SEQ ID NO:4, or biologically active fragment thereof, or a rat NIS protein, e.g., SEQ ID NO:6, or biologically active fragment thereof.

12. The transgenic animal of any of the preceding claims, wherein the nucleic acid comprises a nucleic acid selected from SEQ ID NO:1, SEQ ID NO:3, or SEQ ID NO:5.

13. The transgenic animal of any of the preceding claims, wherein the stably integrated nucleic acid further comprises one or more reporter genes, e.g., fluorescent protein, e.g., GFP, e.g., luminescence generating enzyme, e.g., luciferase.

14. The transgenic animal of claim 13, wherein the one or more reporter genes is under control of the same promoter region that directs expression of the NIS protein.

15. The transgenic animal of claim 13, wherein the one or more reporter genes is under control of a different promoter region that directs expression of the NIS protein.

16. The transgenic animal of any of the preceding claims, wherein the nucleic acid comprises an peptide cleavage element, e.g., an autocatalytic peptide cleavage element, e.g., P2A, T2A.

17. The transgenic animal of any of the preceding claims, wherein the animal is a mouse.

18. The transgenic animal of any of the preceding claims, wherein iodine or technetium uptake is increased relative to a wild-type animal in one or more tissues, e.g., tissues that are not thyroid gland epithelium, gastric epithelium, nasolacrimal duct epithelium, or lactating mammary gland epithelium.

19. The transgenic animal of any of the preceding claims, wherein a NIS protein, or biologically active fragment thereof, is expressed, and wherein the transgenic animal is fertile and passes to its offspring the nucleic acid encoding the NIS protein, or biologically active fragment thereof.

20. The transgenic animal of any of the preceding claims, wherein the transgenic animal is fertile and passes to its offspring the nucleic acid comprising a nucleic acid selected from SEQ ID NO:1, SEQ ID NO:3, or SEQ ID NO:5.

21. The transgenic animal of any of the preceding claims, wherein the animal is heterozygous for the stably integrated nucleic acid, e.g., a founder animal.

22. The transgenic animal of any of the preceding claims, wherein the animal is homozygous for the stably integrated nucleic acid.

23. An isolated cell from the transgenic animal of any of the preceding claims.

24. The isolated cell of claim 23, wherein the isolated cell is a somatic cell.

25. The isolated cell of claim 23, wherein the isolated cell is a germ cell.

26. The cell of any of claims 23-25, wherein the cell ectopically expresses the NIS protein or biologically active fragment thereof.

27. A founder transgenic animal that is hemizygous for somatic and germ cells comprising a stably integrated nucleic acid for ectopic protein expression in transgenic animal cells of a sequence encoding a sodium iodide symporter (NIS) protein or biologically active fragment thereof.

28. A method of making a transgenic animal comprising crossing two founder animals of claim 27.

29. A progeny animal resulting from the cross of claim 28.

30. An in vivo method of detecting NIS activity in a transgenic animal, comprising:
administering to a transgenic animal of any of the preceding claims a detectable substrate of NIS; and
detecting the detectable substrate in the animal.

31. The in vivo method of claim 30, wherein the NIS activity is ion transport e.g., anion uptake into cells, cation export from cells.

32. The in vivo method of claim 30 or 31, wherein the detectable substrate is a detectable anion, e.g., radiolabeled iodine or technetium anion.

33. The in vivo method of any of claims 30-32, wherein the detecting comprises imaging or visualizing the detectable substrate in the animal.

34. The method of any of claims 30-33, wherein the substrate is detected by single photon emission computed tomography (SPECT), Positron Emission Tomography (PET), Magnetic Resonance Imaging (MRI) or scintigraphy.

35. The method of any of claims 30-34, wherein the animal is living at the time of detecting the substrate of NIS.

36. The method of any of claims 30-35, wherein the administering and detecting are performed at more than one timepoint, e.g., 2, 3, 4, 5, 6, 7, 8, 9, 10 or more timepoints, in the same animal.

37. The method of any of claims 30-36, wherein the substrate of NIS is detected at more than one timepoint, e.g., 2, 3, 4, 5, 6, 7, 8, 9, 10 or more timepoints, after administration of the detectable substrate in the same animal.

38. The method of any of claims 30-37, wherein the resolution of the substrate detection is high resolution, e.g., about 0.5 to about 2 mm.

39. The method of any of claims 30-38, wherein the sensitivity of the substrate detection is high sensitivity, e.g., picomolar to nanomolar detection sensitivity.

40. The method of any of claims 30-39, further comprising administering an agent, e.g., a pharmacologic agent, to the transgenic animal, e.g., before, after or simultaneously with the detectable substrate, e.g., before, after or simultaneously with the detecting of the substrate.

41. A method of generating an NIS transgenic animal model disease model, the method comprising:
crossing the transgenic animal of any one of claim 1-22 or 27 with an animal of the same species having a genetic predisposition for development of a disease, disorder or condition,
thereby generating one or more progeny.

42. The method of claim 41, wherein the transgenic animal is selected from a mouse, rat or rabbit.

43. The method of claim 41 or 42, wherein the disease, disorder or condition is selected from cancer, heart disease, hypertension, metabolic and hormonal disorders, diabetes, obesity, osteoporosis, glaucoma, skin pigmentation diseases, blindness, deafness, neurodegenerative disorders (e.g., CNS demyelinating diseases, CNS injury, Amyotrophic lateral sclerosis (ALS), Huntington's disease, Alzheimer's disease, Parkinson's disease, diabetic neuropathy, stroke, idiopathic inflammatory demyelinating disease, multiple sclerosis (MS), optic neuritis (e.g., acute optic neuritis), neuromyelitis optica (NMO), leukodystrophies, vitamin B12 deficiency, progressive multifocal leukoencephalopathy (PML), encephalomyelitis (EPL), acute disseminated encephalomyelitis (ADEM), central pontine myelolysis (CPM), Wallerian Degeneration, adrenoleukodystrophy, Alexander's disease, Pelizaeus Merzbacher disease (PMZ), traumatic glaucoma, periventricular leukomalatia (PVL), or transverse myelitis), psychiatric disturbances (e.g., anxiety or depression), or birth defects (e.g., cleft palate or anencephaly).

44. A progeny animal resulting from the cross of any of claims 41-43.

45. A method of generating an NIS transgenic animal model disease model, the method comprising:
administering to the transgenic animal of any one of claim 1-22 or 27 a compound or a treatment to induce disease;
administering to the animal a detectable substrate of NIS; and
detecting the detectable substrate in the animal at one or more predetermined time intervals.

46. The method of claim 45, wherein the compound is a pharmaceutical compound.

47. The method of claim 45 or 46, wherein the treatment is exposure to a disease inducing agent.

48. The method of any of claims 45-47, wherein the detectable substrate is a detectable anion, such as radiolabeled iodine or technetium anion.

49. The method of any of claims 45-48, wherein the detecting comprises imaging or visualizing the detectable substrate in the animal.

50. A method of evaluating a candidate disease therapy comprising:
administering to an animal of any one of claim 1-22, 27 or 44 a test compound;
administering to the animal a detectable substrate of NIS; and
detecting a change in the detectable substrate in the animal at one or more predetermined time intervals;
thereby identifying a test compound as a candidate disease therapy.

51. A method of monitoring disease progression, comprising:
obtaining a disease model animal by the method of any of claims 41-43 or claims 45-47;
administering to the animal a detectable substrate of NIS; and
detecting the detectable substrate in the animal at one or more predetermined time intervals;
thereby monitoring disease progression in the animal.

52. The method of claim 50 or 51, wherein the detectable substrate is a detectable anion, such as radiolabeled iodine or technetium anion.

53. The method of any of claims 50-52, wherein the detecting comprises imaging or visualizing the detectable substrate in the animal.

54. The method of any of claims 50-53, wherein the substrate is detected by single photon emission computed tomography (SPECT), Positron Emission Tomography (PET), Magnetic Resonance Imaging (MRI) or scintigraphy.

55. The method of any of claims 50-54, wherein the animal is living at the time of detecting the substrate of NIS.

56. The method of any of claims 50-55, wherein the administering and detecting are performed at more than one timepoint, e.g., 2, 3, 4, 5, 6, 7, 8, 9, 10 or more timepoints, in the same animal.

57. The method of any of claims 50-56, wherein the substrate of NIS is detected at more than one timepoint, e.g., 2, 3, 4, 5, 6, 7, 8, 9, 10 or more timepoints, after administration of the detectable substrate in the same animal.

58. The method of claim 50, wherein the test compound is administered at more than one timepoint, e.g., 2, 3, 4, 5, 6, 7, 8, 9, 10 or more timepoints.
